(19) United States
(12) Patent Application Publication
(10) Pub. No.: US 2026/0226486 A1
(43) Pub. Date: Aug. 6, 2026

(54) COMPOSITIONS AND METHODS FOR CONTROLLING T-DNA COPY NUMBER IN TRANSFORMED PLANTS

(71) Applicant: YALE UNIVERSITY, New Haven, CT (US)

(72) Inventors: Yannick JACOB, New Haven, CT (US); Lauren DICKINSON, New Haven, CT (US); Wenxin YUAN, New Haven, CT (US)

(21) Appl. No.: 19/150,200

(22) PCT Filed: Jan. 24, 2024
(86) PCT No.: PCT/US2024/012811

§ 371 Date: Jul. 23, 2025

Related U.S. Application Data
(60) Provisional application No. 63/481,276, filed on Jan. 24, 2023.

(51) Int. Cl. C12N 15/82; C12N 9/22; C12N 15/11

(52) U.S. Cl. CPC C12N 15/8213; C12N 9/226; C12N 15/11; C12N 15/8205; C12N 2310/20

(57) ABSTRACT

Methods of controlling the copy number of one or more genes introduced into a transgenic plant cell, transgenic plants/plant part are disclosed. The method of increasing the gene copy number of a gene introduced into a transgenic plant cell, plant or plant part include contacting the plant cell, plant or plant part with a vector containing a construct which includes an expression cassettes containing multiple pairs of DNA repeats and one or more gene of interest to be introduced into a plant cell under conditions effective for uptake of the vector into the plant cell or a cell in the plant/plant part. Transgenic plant cell, or plants/plant part including one or more than one copies of an exogenous nucleic acid encoding a gene of interest, preferably integrated into the genome of the plant cell, are also disclosed.

### CROSS-REFERENCE TO RELATED APPLICATIONS

This application claims the benefit of and priority to U.S. Provisional Application No. 63/481,276 filed Jan. 24, 2023, the entire content of which is incorporated herein by reference for all purpose in its entirety.

### STATEMENT REGARDING FEDERALLY SPONSORED RESEARCH

This invention was made with government support under Grant #R35GM128661 awarded by National Institutes of Health. The government has certain rights in the invention.

### REFERENCE TO THE SEQUENCE LISTING

The Sequence Listing XML submitted as a file named “YU_8518_PCT_ST26.xml” and having a size of 42,106 bytes is hereby incorporated by reference pursuant to 37 C.F.R. § 1.834(c)(1).

### FIELD OF THE INVENTION

The disclosed invention is generally in the field of transgenic plants and specifically in the area of increasing gene copy number in transgenic plants.

### BACKGROUND OF THE INVENTION

Plant transformations, including CRISPR-based gene editing in plants, frequently rely on the integration of an Agrobacterium-derived T-DNA carrying the editing components into the genome. Studies in plants have shown that in vivo levels of Cas9-sgRNA and repair templates correlate positively with the frequencies of targeted mutagenesis and gene targeting, respectively. Therefore, increasing T-DNA copy number in cells may positively impact gene editing and other biotechnological applications that depend on Agrobacterium-mediated plant transformation.

It is an object of the present invention to provide compositions for increasing gene copy number of one or more genes introduced into plant cells to form transgenic plant cells.

It is also an object of the present invention to provide methods for increasing gene copy number of one or more genes introduced into plant cells to form transgenic plant cells.

It is still an object of the present invention to provide transgenic plants and plant material with increased gene copy number of one or more genes introduced into the transgenic plants and plant material.

### SUMMARY OF THE INVENTION

Methods of increasing gene copy number of one or more genes introduced into a plant cell, plant/plant part, thereby forming a transgenic plant cell, plant/plat part are disclosed. The gene/polynucleotide/nucleic acid introduced into a plant cell plant cell, plant/plant part is referred to herein as “exogenous gene/polynucleotide/nucleic acid” in the sense that it originates outside the plant cell plant cell, plant/plant part. However, the exogenous/polynucleotide/nucleic acid can include a heterologous or endogenous sequence. In one preferred embodiment, the exogenous polynucleotide/nucleic acid is a T-DNA. Transgenic plant cell, transgenic plants/plant part with increased exogenous gene copy number are also disclosed. The transgenic plant cell, or plants/plant part includes more than one copies of exogenous nucleic acid encoding a gene of interest, preferably integrated into the genome of the plant cell, and optionally, multiple pairs of DNA repeats with an linker sequence of defined length separating the gene of interest and the DNA repeats. In one embodiment, the DNA repeats are preferably provided by a retrotransposon containing LTR (long terminal repeats), such as ONSEN, Copia13, copia2l, EVD or GP3-1. In some forms, the DNA repeats have the same length and GC content as the LTR of ONSEN (440 bp), Copiat3 (158 bp), copia2t (119 bp), EVD (406 bp) and GP3-1 (384 bp). In some forms the DNA repeats are 250-600 bp in length and have the same length and GC content as ONSEN, Copia13, copia2l or GP3-1. In one embodiment, the gene of interest is a CAS endonuclease such as CAS9 and one or more guide polynucleotide sequences, for example, single guide RNA (sgRNA) designed to guide the CAS endonuclease to a specific gene of interest in the plant cell, plant/plant part.

In a preferred embodiment, the more than one copy of the gene of interest are present as concatemers (i.e., in a chain or series) in the genome of the transgenic plant cell, plant/plant part.

The method of increasing gene copy number of a gene of interest introduced into a transgenic plant cell, plant or plant part includes contacting the plant cell, plant or plant part with a vector containing a construct which includes an expression cassette containing multiple pairs of DNA repeats (with an optional linker sequence of defined length separating individual repeats) and a gene of interest to be introduced into a plant cell under conditions effective for uptake of the vector into the plant cell or a cell in the plant/plant part.

### BRIEF DESCRIPTION OF THE DRAWINGS

FIG. 1A is a schematic of the structure of the endogenous ONSEN RT and the modified RT used in the ONSEN RT-based vector. LTR: long terminal repeat; gag: gag-like protein; PR: protease; int: integrase; RT/RH: reverse transcriptase/RNase H; PPT: polyurine tract. FIG. 1B is a schematic representation of No RT construct (adapted from [11]) and ONSEN RT construct. Arrows indicate the primers used for real time quantification shown in FIGS. 1C and 1D. LB: left border; RB: right border. FIG. 1C shows quantitative PCR of ALS in Col, No RT and ONSEN RT T1 plants. Each dot represents an individual plant. Horizontal bars indicate the median. *P<0.001 (Mann-Whitney U test). FIG. 1D shows DNA-qPCR of ALS, pea3A(T) and NPTII FIG. 1E shows the transgene copy number as determined by whole genome sequencing analysis. FIG. 1F shows the T-DNA insertion sites in No RT and ONSEN RT T1 plants.

FIG. 2A-2F show the repetitive nature of LTRs induces T-DNA amplification. FIG. 2A is a table of the characteristics of other retrotransposons used to make RT-based constructs. FIG. 2B is a dot plot of quantitative PCR of ALS in plant transformed with various RT-based constructs. Each dot represents an individual T1 plant. Horizontal bars indicate the median. ns=not significantly different (Kruskal-Wallis ANOVA followed by Dunn's test). FIG. 2C is a schematic representation of deletion constructs used in FIG. 2D. FIG. 2D is a dot plot of quantitative PCR of ALS in plant transformed with RT deletion constructs. Each dot represents an individual T1 plant. Horizontal bars indicate the median. *P<0.05, **P<0.01, ***P<0.001, ****P<0.0001 (Kruskal-Wallis ANOVA followed by Dunn's test). FIG. 2E is a schematic representation of random repeat construct used in FIG. 2F. FIG. 2F is a dot plot of quantitative PCR of ALS in plant transformed with RT deletion constructs. Each dot represents an individual T1 plant. Horizontal bars indicate the median. **P<0.01, ***P<0.001, ns=not significantly different (Kruskal-Wallis ANOVA followed by Dunn's test).

FIG. 3A is a dot plot of relative ALS quantification of No RT- and ONSEN RT-transformed Agrobacterium. Each dot represents a liquid culture from an independent colony. FIG. 3B shows the log 2 ratio between the number of reads corresponding to the genomic regions surrounding the T-DNA insertion sites compared to Col. The grey box represents the interval where 95% of copy number values from the Arabidopsis genome are present. FIGS. 3C-3F are dot plots of the relative ALS quantification in mutant backgrounds of the HR (FIG. 3C), NHEJ (FIG. 3D) and TMEJ (FIG. 3E) pathways, and of DNA damage-induced kinases (FIG. 3F). Each dot represents an individual T1 plant. Horizontal bars indicate the median. *P<0.01, ns=not significantly different (Kruskal-Wallis ANOVA followed by Dunn's test).

FIG. 4A is a bar graph of the percentage of indels in the CRY2 gene of DNA extracted from leaves of individual T1 plants transformed with No RT and ONSEN RT constructs containing CRY2 guide RNA #1 (FIG. 5). FIG. 4B is a schematic representations of constructs used for ALS gene targeting. FIG. 4C is a schematic of the In planta GT approach with programmed T-DNA amplification. FIG. 4D is a bar graph of the percentage of IM-resistant T2 plants from individual T1 parents transformed with No RT or ONSEN RT constructs. FIG. 4E is a table summary of gene targeting events using No RT and ONSEN RT constructs. FIG. 4F is a bar graph of the relative ALS quantification of ONSEN-RT T1 plants (gray) in relationship to percentage of IM-resistance for associated T2 plants (red). Frequency of ALS gene targeting events using No RT and ONSEN RT constructs.

FIG. 5 is a DNA FISH analysis showing quantitative PCR of ALS in Col and T1 plants used for the FISH experiment. Bars indicate the mean of DNA-qPCR using DNA from different leaves. SD is shown.

FIGS. 6A and 6B show whole genome sequencing analysis of T-DNA insertions. FIG. 6A is a bar graph of quantitative PCR of ALS in the No RT and ONSEN RT T1 plants used for whole genome sequencing (FIGS. 1E and 1F). FIG. 6B is a collection of locus diagrams for the identified T-DNA insertions. For each insertion, top lines represent unaltered genomic sequence with annotated genes. Arrows represent insertion points. The bottom lines show the borders of the insertion in more detail, with the identified vector or T-DNA components shown. Dashed lines represent contiguous T-DNA-associated cassettes. Shades of grey bars indicate binary vector sequence, LB bars and pea3A terminator sequence bars indicate the 5′ end of the T-DNA construct (though it may be in 5′ or 3′ orientation in the plant genome). Light gray bars next to darker gray bars are junction filler sequences, and the bar representing NPTII sequence is shown.

FIGS. 7A and 7B show repetitive gRNA genes do not contribute to T-DNA amplification. FIG. 7A is a schematic representation of the single gRNA gene deletion from the No RT vector. FIG. 7B is a dot plot of quantitative PCR of ALS in Col, and in T1 plants transformed with the No RT plasmid and the No RT plasmid with a gRNA gene deletion. Each dot represents and individual plant. ns=not significantly different (Mann-Whitney U test).

FIGS. 8A-8C show concatenated T-DNA junctions support the involvement of TMEJ in T-DNA amplification. FIG. 8A shows multiple sequence alignments of junctions between two T-DNA sequences grouped by orientation of the two sequences (RB-LB, LB-LB or RB-RB). Reference sequences were constructed assuming T-DNA molecules began and ended immediately upstream of the endonuclease recognition sequence (5′-CAGGATATATT-3′) (SEQ ID NO:8)^{53,54}. Filler sequences are in red and sequences consistent with microhomology-associated deletions are underlined. If filler sequences have a similar sequence nearby, they (and the nearby sequence) are also underlined. Asterisks indicate identical junctions occurring in independent plants. FIG. 8B is a depiction of T-DNA junctions with another T-DNA or binary vector sequence not immediately internally adjacent to the LB or RB. FIG. 8C is a classification of RB-LB, LB-LB and RB-RB sequences for each T1 line following a procedure previously described55. NHEJ (<4 bp deletions and <5 bp insertions), insertions (≥5 bp with any deletion), Non-Microhomology (Non-MH; ≥4 bp deletion or <5 bp insertions with microhomologies <2), and Microhomology (MH; ≥4 bp deletion with microhomologies ≥2). The latter three are associated with DNA polymerase theta.

FIGS. 9A and 9B show that T-DNA amplification is not DNA replication-dependent. FIG. 9A is a dot plot of quantitative PCR of ALS in Col, atxr5/6, and tsk plants transformed with the ONSEN RT construct. Each dot represents an individual T1 plant. FIG. 9B is a bar graph of quantitative PCR results from ALS in DNA extracted from leaves of T1 plants at 16 days and 30 days after germination.

FIGS. 10A-10D show programmed T-DNA amplification increases the efficiency of mutagenesis and gene targeting. FIG. 10A is a schematic of the gene structure of CRY2, with targeting sequences (SEQ ID NO:5 (left sequence); SEQ ID NO:6 (middle sequence) and SEQ ID NO:7 (right sequence). Gray bars represent exons, and darker gray bars represent regions targeted by gRNAs. FIGS. 10B and 10C show the percentage of CRISPR-induced indels in individual T1 lines transformed with either the ONSEN RT or the No RT construct (FIG. 4A). Constructs carried either guide #2 (FIG. 10B) or guide #3 (FIG. 10C). FIG. 10D shows the relative ALS quantification of ONSEN-RT T1 plants (gray) in relationship to percentage of GT rates in the T2 generation (red).

FIG. 11A is a schematic representation of the replication of endogenous retrotransposons (RTs). Through RTs are typically silenced, activation leads to transcription to form mRNA. The mRNA encodes the proteins necessary for replication, including proteins that surround the mRNA to form a virus-like particle, a reverse transcriptase that will reverse transcribe the mRNA to form a new DNA copy, and integrase, which will allow the nascent copies to integrate into new genomic sites. FIG. 11B is a schematic representation of the function of traditional RT-based vectors. Incorporation of the desired exogenous DNA sequence will disrupt the sequence that codes the necessary replication proteins and prevent autonomous replication of the transformed T-DNA sequence. To avoid this issue, “helper” RTs containing the full RT sequence are also transformed. The autonomous helper RTs can produce proteins compatible with reverse transcription and transportation of the nonautonomous vector RT. The RT vectors replicated via helper proteins can then be integrated into new genomic locations or remain extrachromosomal within the nucleus. FIG. 11C is a schematic representation of the application of the programmed T-DNA amplification. In this case, exogenous DNA flanked by the RT backbone leads to multiple copies of the vector arranged in a concatenated form (i.e., tandem duplication) at the transformation site. This allows for multiple desired DNA copies without transcription, translation, reverse transcription, and transportation, and the copies can be easily removed through backcrossing to wildtype (untransformed) plants or controlled exonuclease excision. RT-mediated amplification using this tandem duplication mechanism occurs in transformation of plant species when RT-derived sequences (or equivalent sequences like engineered DNA repeats) are included on the expression vector. For example, RT-mediated amplification using this tandem duplication mechanism occurs in the context of Agrobacterium-mediated transformation of plant species when RT-derived sequences (or equivalent sequences like engineered DNA repeats) are included on the T-DNA region of a tumor-inducing plasmid

### DETAILED DESCRIPTION OF THE INVENTION

### I. Definitions

The term “agroinfiltration” as used herein refers to a method in plant biology to transfer genetic cassettes from Agrobacterium into a plant. In the method a suspension of Agrobacterium tumefaciens is injected into a plant leaf, where it transfers the desired gene to plant cells. The benefit of agroinfiltration when compared to traditional plant transformation is speed and convenience.

The term “cell” refers to a membrane-bound biological unit capable of replication or division.

The term “construct” refers to a recombinant genetic molecule having one or more isolated polynucleotide sequences. Genetic constructs used for transgene expression in a host organism include a series of cassettes including units with (in the 5′-3′ direction), a promoter sequence; a sequence encoding a gene of interest; and a termination sequence. The construct may also include selectable marker gene(s) and other regulatory elements for expression.

As used herein, a “cultivar” refers to a cultivated variety.

As used herein, the term “derivative species, germplasm or variety” refers to any plant species, germplasm or variety that is produced using a stated species, variety, cultivar, or germplasm, using standard procedures of sexual hybridization, recombinant DNA technology, tissue culture, mutagenesis, or a combination of any one or more said procedures.

The term “expression” as used herein refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “expression vector” refers to a vector that includes one or more expression control sequences.

The term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. In eukaryotes the term “expression vector” refers to a vector that includes one or more expression control sequences regardless of the origin of the sequence (prokaryote or eukaryote).

The term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

As used herein, “germplasm” refers to one or more phenotypic characteristics, or one or more genes encoding said one or more phenotypic characteristics, capable of being transmitted between generations.

The term “genome” as used herein, referring to a plant cell encompasses not only chromosomal DNA found within the nucleus, but organelle DNA found within subcellular components (e.g., mitochondria, or plastid) of the cell.

The term “guide polynucleotide” as used herein refers to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence).

As used herein the term “heterologous” means from another host. The other host can be the same or different species.

The term “plant” is used in its broadest sense. It includes, but is not limited to, any species of woody, ornamental or decorative crop or cereal, and fruit or vegetable plant. It also refers to a plurality of plant cells that are largely differentiated into a structure that is present at any stage of a plant's development. Such structures include, but are not limited to, a fruit, shoot, stem, leaf, flower petal, etc.

The term “plant cell” refers to a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

The term “plant cell culture” refers to cultures of plant units such as, for example, protoplasts, cell culture cells, cells in plant tissues, pollen, pollen tubes, ovules, embryo sacs, zygotes and embryos at various stages of development.

The term “plant material” refers to leaves, stems, roots, flowers or flower parts, fruits, pollen, egg cells, zygotes, seeds, cuttings, cell or tissue cultures, or any other part or product of a plant.

The term “plant organ” refers to a distinct and visibly structured and differentiated part of a plant such as a root, stem, leaf, flower bud, or embryo.

As used herein, “plant part” or “part of a plant” can include, but is not limited to cuttings, cells, protoplasts, cell tissue cultures, callus (calli), cell clumps, embryos, stamens, pollen, anthers, pistils, ovules, flowers, seed, petals, leaves, stems, and roots.

The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture. The term “plant part” as used herein refers to a plant structure, a plant organ, or a plant tissue.

The term “promoter” refers to a regulatory nucleic acid sequence, typically located upstream (5′) of a gene or protein coding sequence that, in conjunction with various elements, is responsible for regulating the expression of the gene or protein coding sequence.

As used herein, the term “progenitor” refers to any of the species, varieties, cultivars, or germplasm, from which a plant is derived.

The term “transgenic plant/cell” refers to a plant/cell that contains recombinant genetic material which has been introduced into the plant/cell in question (or into progenitors of the plant) by human manipulation. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems etc.

A “transgene” as used herein refers to an artificial gene, manipulated in the molecular biology lab that incorporate all appropriate elements critical for gene expression generally derived from a different species.

The terms “transformed,” “transgenic,” “transfected” and “recombinant” refer to a host organism such as a bacterium or a plant into which a exogenous nucleic acid molecule has been introduced.

The term “vector” refers to a replicon, such as a plasmid, phage, or cosmid, into which another DNA segment may be inserted so as to bring about the replication of the inserted segment. The vectors can be expression vectors.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

Recitation of ranges of values herein are merely intended to serve as a shorthand method of referring individually to each separate value falling within the range, unless otherwise indicated herein, and each separate value is incorporated into the specification as if it were individually recited herein.

The use of any and all examples, or exemplary language (e.g., “such as”) provided herein, is intended merely to better illuminate the description and does not pose a limitation on the scope of the description unless otherwise claimed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Compositions

Genetically modified constructs containing a gene of interest to be introduced into a plant, plant vectors including the constructs, as well as plant/plant parts genetically engineered using the disclosed constructs and vectors are disclosed.

### A. Genetically Modified Constructs Including Gene of Interest

Nucleic acid constructs which include expression cassettes containing multiple pairs of DNA repeats (with an optional linker sequence of defined length separating individual repeats) and a gene of interest to be introduced into a plant cell are disclosed. The DNA repeats are preferably provided by a retrotransposon containing LTR (long terminal repeats). In one embodiment, the random repeat contains the following sequence. AGTATGTGTTTTTCCTTCAAGAGGTATGTGGGTGCGTGAACAAATGAATAGTATACGTATTTACTCG ATGTGTTTAGTTTCTCATATTTCTCCTGGAAATTAAAAAAATATTTTATATCCAAATGAAAAACCGT TTTACGGAATGAATCTACATTATTACCTGTATAAAAAAATCAATATAGCTAAGGACAAAAGCGACT TTAAATTCTAATTATTGATTTTAGCGAAAAAGTTTCATTTAGAAAGCAGATATTGATTGACACGATT TAATAGAACGTTTGAGGATTAGATTAAATTAAATAGTTTAATATTGATATATCTGGCTTTAAAATTT AGTATAGTATATTAATCGGAGACGAATTAAAAACACAAATTTTCAAAATCAAACGAGCTCATTACA TCAATTAATTTTGATATATTATGTGAACAATGTTTTAAAG (SE ID NO:1) or a variant thereof having more than 90%, 91%, 92%, 93%, 94%, 95%, 96%, 97%, 98%, or 99% identical to the sequence of SEQ ID NO. 1.

In one embodiment, the construct contains operatively linked in the 5′ to 3′ direction, the first copy of a DNA repeat, an optional linker; one or more nucleic acid sequences encoding one or more genes of interest to be introduced into a plant; an optional linker and the second copy of the DNA repeat.

### (i) Retrotransposons

Retrotransposons are major components of plant and animal genomes. They amplify by reverse transcription and reintegration into the host genome, but their activity is usually epigenetically silenced. In plants, genomic copies of retrotransposons are typically associated with repressive chromatin modifications installed and maintained by RNA-directed DNA methylation. Long Terminal Repeat (LTR) retrotransposons are ubiquitous components of plant genomes. Because of their copy-and-paste mode of transposition these elements tend to increase their copy number while they are active. A common feature of LTR-retrotransposons is the presence of two direct repeats flanking the central region of the element (these repeats are the 5′ LTR and 3′ LTR). LTRs can be subdivided into three regions: U3, R, and U5. U3 contains the enhancer and promoter sequences that drive viral transcription. R domain encodes the 5′ capping sequences (5′ cap) and the polyA (pA) signal. The process of reverse transcription renders the LTRs identical at the moment of integration of a new retrotransposon copy. They flank an internal sequence, which may or not code for the proteins necessary for carrying out retrotransposition. These proteins are encoded in two primary open reading frames (ORFs), which may in some elements be fused into one: the Gag, encoding the structural protein involved in nucleocapsid formation; the Pol, specifying the activities for the reverse transcription and integration of new copies. The Pol is a polyprotein and contains domains for an AP (aspartic protease), responsible for the post-translational processing of the Pol ORF product, RT (reverse transcriptase) and RNAseH, which, as a bifunctional polypeptide, carries out reverse transcription and IN (integrase), which inserts the new LTR retrotransposon copy into the genome.

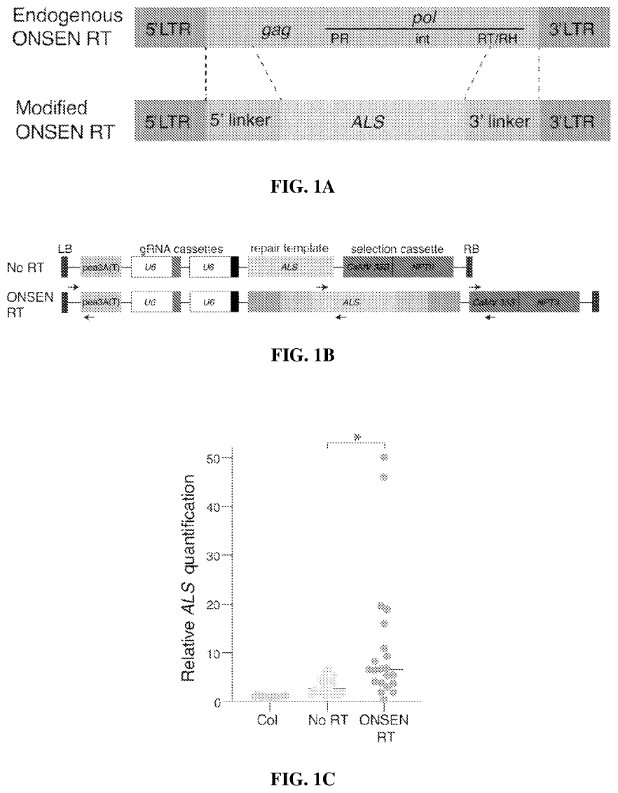

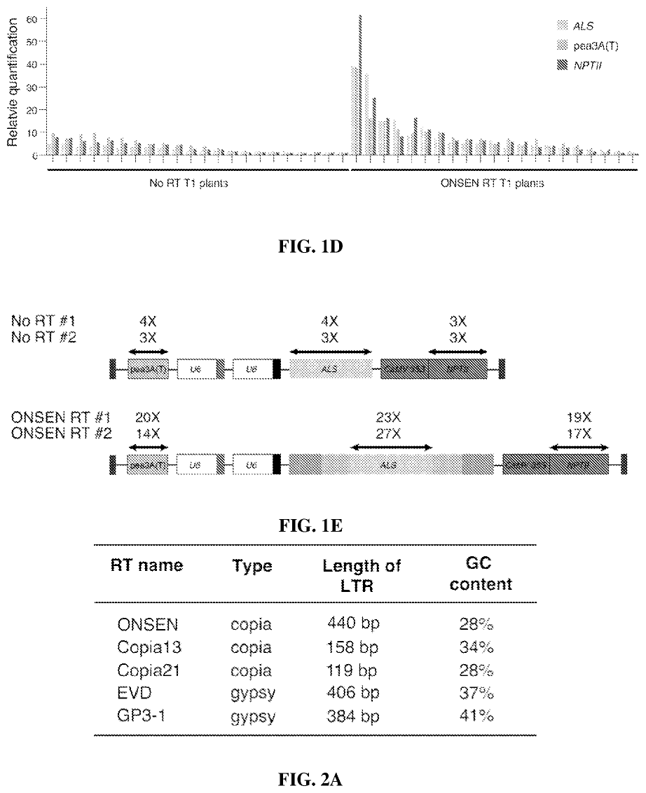

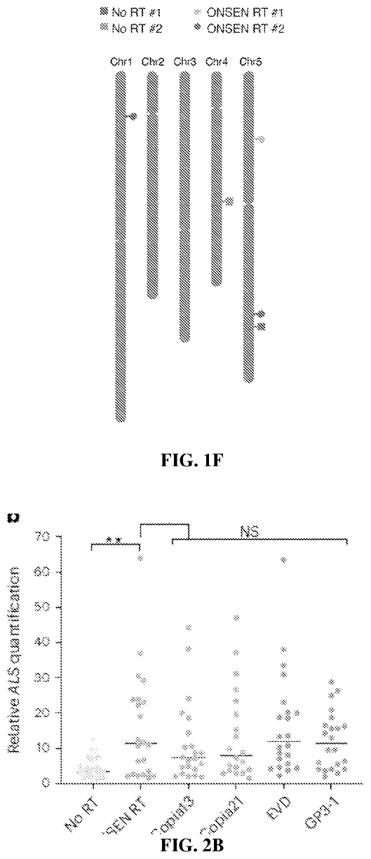

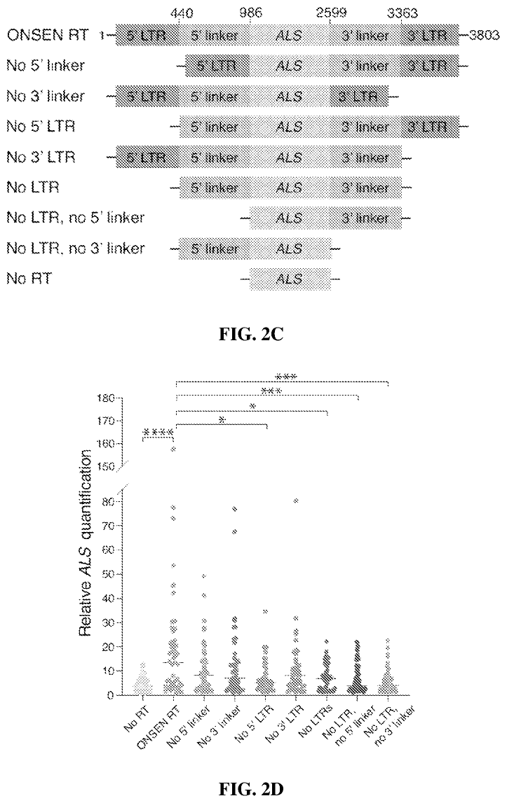

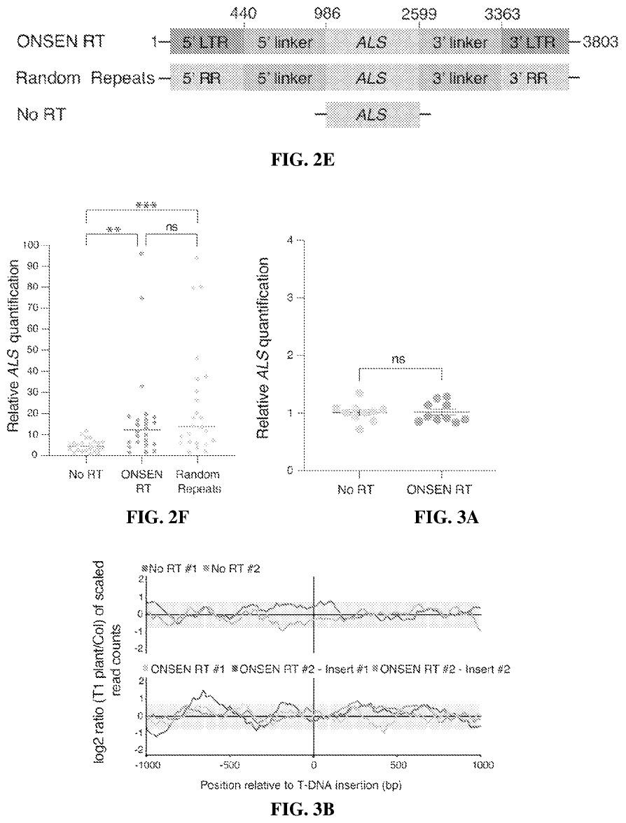

RT used in the disclosed constructs are non-autonomous or rendered non-autonomous. Useful RT include the Ty1-copia elements and the Ty3-gypsy elements which are the two main superfamilies of LTR retrotransposons. Useful retrotransposons include, but are not limited to ONSEN, Copia13, copia2l, EVD and GP3-1, rider family of RT (Benoit, et sl., PLoS Genet. 15(9): e1008370. (2019). ONSEN, is an LTR-copia type retrotransposon in Arabidopsis thaliana. EVADE (EVD) is a retrotransposon of the ATCOPIA93 family. The RT is engineered to replace part of the gag and pol genes with DNA of interest to be inserted into a plant (FIG. 1A). As shown therein, when the retrotransposon construct is created, part of the open reading frame (ORF, containing the gag and pol genes) is removed, to incorporate the desired sequence. The linker sequences are what remains of the ORF, between the retrotransposon repeats and the desired sequence on each side (FIG. 1A). In FIG. 2D, shows that deleting these linker sequences somewhat decreases the level of T-DNA copy number, indicating that they likely play a role in generating high copy numbers.

### (ii) Non-RT DNA Repeats

The nucleic acid constructs can include multiple pairs of DNA repeats (not provided by RT), and/or a linker sequence of defined length separating individual repeats and a gene of interest to be introduced into a plant cell are disclosed. For example, the DNA repeats can have the same length and GC content as the LTR of ONSEN (440 bp), Copia13 (158 bp), copia2l (119 bp), EVD (406 bp) and GP3-1 (384 bp). LTR retrotransposons contain two long-terminal repeats (LTRs), at their ends, typically 250-600 bp in length; thus, the multiple pairs of DNA repeats are selected to have the same length and GC content as an LTR which are known in the art (FIG. 2A). Exemplary sequences are shown below.

(SEQ ID NO: 1)
AGTATGTGTTTTTCCTTCAAGAGGTATGTGGGTGCGTGAACAAATGAATAGTATACGTATT
TACTCGATGTGTTTAGTTTCTCATATTTCTCCTGGAAATTAAAAAAATATTTTATATCCAAATGAAA
AACCGTTTTACGGAATGAATCTACATTATTACCTGTATAAAAAAATCAATATAGCTAAGGACAAAA
GCGACTTTAAATTCTAATTATTGATTTTAGCGAAAAAGTTTCATTTAGAAAGCAGATATTGATTGAC
ACGATTTAATAGAACGTTTGAGGATTAGATTAAATTAAATAGTTTAATATTGATATATCTGGCTTTA
AAATTTAGTATAGTATATTAATCGGAGACGAATTAAAAACACAAATTTTCAAAATCAAACGAGCTC
ATTACATCAATTAATTTTGATATATTATGTGAACAATGTTTTAAAG;
>COPIA 13 LTR (At2g13940)
(SEQ ID NO: 31)
TATTGGGCCATATGTATGGACCTTATATGTTTTGGGCTTTAACTGTAACTTATCGATGAAGTCTCAT
AAACCCTTGTGTAAACCCTAGCAGCTTTGTGTATATAAGCAATTGTATGAATGATCAATAAAGCAA
GTCAGTTCGATATCTAATTCTACAT;
>COPIA 21 LTR (At5g44925)
(SEQ ID NO: 32)
TATAAGGAGTTGTATATATTAAGGATATAATTGTCATTAGTAGTTGTGTATACCCTAGACTAAGTA
CTATATATGTTGTAACACATTCATCATAATAAGACAATTCACTCTCTTATCAC;
>Evade LTR (At5g17125)
(SEQ ID NO: 33)
TATTGATCAAGACTCAAATAAGAAAGGCCTAGTATTGGATATGTACTACAAGAGAGTGGGCCGAA
CATATGAGAAGTCTATGAAGAGCTTCTAGAAGAGGTGAAGGCACACAAATATCTCTTGTAGCCGT
TGAAGAGGACTCACGAGTGTCTTTAGCAACAAGGTCAACCAATGACCAGTCACTCTCATCACCAA
GACCAAGGAACCTCTTCTCTTATGTCTCTTTCAGTCTTACGTTAATTGTAAACAGCCATATTCTCTT
TGTGTGTCTTTAAAAGCATTGTAAACACACAAAGGTTACTATCTAATTCTATCAATATGATTTGGTC
TCATATTCTCTCACATACAAACTCTCTTGTTCTTCATAACTCTCTTAATCTTTAATACAATTCCGCAT
ATTCTTTCA;
and
>GP3-1 LTR (At3g11970):
(SEQ ID NO: 34)
TGATACAGGATTAACGTGTGGTGAGCTGGTGAGCTGACGAGCTGGCGAGCTGGCAATGTGAAGTC
GGATGTTAGGCAAAGTACTTGTGAGCTGGCGAGCTGGCGAGCTGGCAATGCAATGGACGTTAACG
GCGATCGTTATTAACGTGGTTAACGTTTACTTTCTTTACTTTTTTATATAAGAGCAACATGTTATGC
TCATGCTTTCATTATCGAATAATCTGTAATCTTCAGAATTCTCTAGAACCTTTGTAAGTGCTAGATT
TCTCTCACATCGAGAGCTAGCTGAATCATTTGGTGTTTTATGAACATCGATTGATTGTTGGTTTCGA
ATGATAATTGGAGATTTGGATTGTTCTTGTGAATCGCAGGTTTCCCTGTAACA

Useful sequences include sequences having more than 90%, 91%, 92%, 93%, 94%, 95%, 96%, 97%, 98%, or 99% identical to the sequence of SEQ ID NO:1, and 32-34.

### B. Expression Vectors

The nucleic acid construct is operably linked to a promoter, in a suitable expression vector. A nucleic acid sequence or polynucleotide is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading frame. Linking can be accomplished by ligation at convenient restriction sites. If such sites do not exist, synthetic oligonucleotide adaptors or linkers are used in accordance with conventional practice.

The expression vector can be any expression vector suitable for plant transformation, such as a plasmid or a plant viral vector. The terms “plasmid”, “vector” and “cassette” as used herein refer to an extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. A preferred plasmid is a T1 binary plasmid.

Ti-plasmid, short for tumour-inducing plasmid, is an extrachromosomal molecule of DNA found commonly in the plant pathogen Agrobacterium tumefaciens. It is also found in other species of Agrobacterium such as A. rubi, A. vitis and A. rhizogenes. Ti plasmids contain one or more T-DNA regions, sections that are transferred to plant cells during infection. The T-DNA region is the crucial region that gets transferred to the plant cell for infection. It is approximately 15-20 kb in length and is transferred to the plant cell via means of genetic recombination.

### Promoters

The promoters suitable for use in the constructs of this disclosure are functional in plants, which can include a promoter provided by a RT if used in the construct. Plant promoters can be selected to control the expression of the transgene in different plant tissues or organelles for all of which methods are known to those skilled in the art (Gasser & Fraley, Science 244:1293-99 (1989)). In one embodiment, promoters are selected from those of eukaryotic or synthetic origin that are known to yield high levels of expression in plant and algae cytosol. In another embodiment, promoters are selected from those of plant or prokaryotic origin that are known to yield high expression in plastids. In certain embodiments the promoters are inducible. Inducible plant promoters are known in the art. In one embodiment, the promoter is an egg cell-specific promoter.

Some preferred promoters include, but are not limited to the EC1 promoter¹¹:

(SEQ ID NO: 2)
gaataaaagcatttgcgtttggtttatcattgcgtttatacaaggacagagatccactgagctggaatagcttaaaaccattatcagaacaa
aataaaccattttttgttaagaatcagagcatagtaaacaacagaaacaacctaagagaggtaacttgtccaagaagatagctaattatat
ctattttataaaagttatcatagtttgtaagtcacaaaagatgcaaataacagagaaactaggagacttgagaatatacattcttgtatatt
tgtattcgagattgtgaaaatttgaccataagtttaaattcttaaaaagatatatctgatctagatgatggttatagactgtaattttacca
catgtttaatgatggatagtgacacacatgacacatcgacaacactatagcatcttatttagattacaacatgaaatttttctgtaatacat
gtctttgtacataatttaaaagtaattcctaagaaatatatttatacaaggagtttaaagaaaacatagcataaagttcaatgagtagtaaa
aaccatatacagtatatagcataaagttcaatgagtttattacaaaagcattggttcactttctgtaacacgacgttaaaccttcgtctcca
ataggagcgctactgattcaacatgccaatatatactaaatacgtttctacagtcaaatgctttaacgtttcatgattaagtgactatttac
cgtcaatcctttcccattcctcccactaatccaactttttaattactcttaaatcaccactaagctagtaacgcctatcatgaattagctct
actaaatctagcaacctttcaaatttgcagtattgcaggtgtctctgtgtctttaaaatagttgccttatgatttcttcggtttcaagatga
tcaaatagttatagatttcatgctcacacatgctcattagatgtgtacatactttacttacccaaatctattttctcgcaaagattttgatg
gtaaagctgatttggttctattgaactaaatcaaacgagtttcagactgagtgattctaatccggcccattagcccctaaacagacccacta
attacgcagcttttaatagagtaattacacctagtttacccactaaaccactaagcactaattatctcacaatctaatgagcttccctcgta
attacttgggctttcactctaccatttatttgtaacagtcaagtctctactgtctctatataaactctctaaagttaacacacaattctcat
cacaaacaaatcaaccaaagcaacttctactctttcttctttcgaccttatcaatctgttgagaa;

Many plant promoters are publicly known. These include constitutive promoters, inducible promoters, tissue- and cell-specific promoters and developmentally-regulated promoters. Exemplary promoters and fusion promoters are described, e.g., in U.S. Pat. No. 6,717,034, which is herein incorporated by reference in its entirety.

Suitable constitutive promoters for nuclear-encoded expression include, for example, the core promoter of the Rsyn7 promoter and other constitutive promoters disclosed in U.S. Pat. No. 6,072,050; the core CAMV 35S promoter, (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); and ALS promoter (U.S. Pat. No. 5,659,026). Other constitutive promoters include, for example, U.S. Pat. Nos. 5,608,149; 5,608,144; 5,604,121; 5,569,597; 5,466,785; 5,399,680; 5,268,463; 5,608,142.

“Tissue-preferred” promoters can be used to target a gene expression within a particular tissue such as seed, leaf or root tissue. Tissue-preferred promoters include Yamamoto et al. (1997) Plant J. 12(2)255-265; Kawamata et al. (1997) Plant Cell Physiol. 38(7):792-803; Hansen et al (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al (1996) Plant Physiol. 112(2):525-535; Canevascini et al. (1996) Plant Physiol. 112(2):513-524; Yamamoto et al. (1994) Plant Cell Physiol. 35(5):773-778; Orozco et al. (1993) Plant Mol. Biol. 23(6):1129-1138; Matsuoka et al. (1993) Proc Natl. Acad. Sci. USA 90(20):9586-9590; and Guevara-Garcia et al. (1993) Plant J. 4(3):495-505.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108. Such seed-preferred promoters include, but are not limited to, Cim1 (cytokinin-induced message); cZ19B1 (maize 19 kDa zein); milps (myo-inositol-1-phosphate synthase); and ce1A (cellulose synthase). Gama-zein is a preferred endosperm-specific promoter. Glob-1 is a preferred embryo-specific promoter. For dicots, seed-specific promoters include, but are not limited to, bean β-phaseolin, napin β-conglycinin, soybean lectin, cruciferin, oleosin, the Lesquerella hydroxylase promoter, and the like. For monocots, seed-specific promoters include, but are not limited to, maize 15 kDa zein, 22 kDa zein, 27 kDa zein, g-zein, waxy, shrunken 1, shrunken 2, globulin 1, etc. Additional seed specific promoters useful for practicing this invention are described in the Examples disclosed herein.

Leaf-specific promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and may be selected from the many available from the literature or isolated de novo from various compatible species. See, for example, Hire et al. (1992) Plant Mol. Biol. 20(2): 207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):1 1′-22 (full-length cDNA clone encoding cytosolic glutamine synthetase (GS), which is expressed in roots and root nodules of soybean). See also U.S. Pat. Nos. 5,837,876; 5,750,386; 5,633,363; 5,459,252; 5,401,836; 5,110,732; and 5,023,179.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize 1n2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. Proc. Natl. Acad. Sci. USA 88:10421-10425 (1991) and McNellis et al. Plant J. 14(2):247-257(1998)) and tetracycline-inducible and tetracycline-repressible promoters (see, for example, Gatz et al. Mol. Gen. Genet. 227:229-237 (1991), and U.S. Pat. Nos. 5,814,618 and 5,789,156), herein incorporated by reference in their entirety.

### Transcription Termination Sequences

At the extreme 3′ end of the transcript of the transgene, a polyadenylation signal can be engineered. A polyadenylation signal refers to any sequence that can result in polyadenylation of the mRNA in the nucleus prior to export of the mRNA to the cytosol, such as the 3′ region of nopaline synthase (Bevan, et al. Nucleic Acids Res. 1983, 11, 369-385). Examples of other terminator sequences include:

rbcS-E9 terminator sequence¹¹:
(SEQ ID NO: 36)
agagctttcgttcgtatcatcggtttcgacaacgttcgtcaagttcaatgcatcagtttcattgcgcacacaccagaatcctact
gagtttgagtattatggcattgggaaaactgtttttcttgtaccatttgttgtgcttgtaatttactgtgttttttattcggttttcgctat
cgaactgtgaaatggaaatggatggagaagagttaatgaatgatatggtccttttgttcattctcaaattaatattatttgttttttctctt
atttgttgtgtgttgaatttgaaattataagagatatgcaaacattttgttttgagtaaaaatgtgtcaaatcgtggcctctaatgaccgaa
gttaatatgaggagtaaaacactigtagtigtaccattatgcttattcactaggcaacaaatatattttcagacctagaaaagctgcaaatg
ttactgaatacaaglaactatttttttaaaaaatttgtcctcttgtgttttagacatttatgaactttcctttatgtaattttccagaatcc
ttgtcagattctaatcattgctttataattatagttatactcatggatttgtagttgagtatgaaaatattttttaatgcattttatgactt
gccaattgattgacaac:
CLAVATA 3 terminator sequence¹¹:
(SEQ ID NO: 37)
taatctcttgttgctttaaattatttcatattgtaaattactttctgctttatcggttttaccatttcgggagtcttttttgtgtgcaatct
gtaagcttgtagtttcatgaaagtgaatgtaagatatgcattacgtttgttgctgaagtgaatgtaagatacgcactattatatctcatgat
ttgtttcgtttgtctaagaaaaccctcttaaaacgaagatgtctatagcattacgtttctatttccatataatacgttaaaatttatggttt
ttacgtataaaatgcaaaataaagacacaagtatatctccaaagcaatgtaccgttgggaaaatttattagtacgttttcaattgtcaatgc
aaataattaatggatgtgatagtcacaattaaacatacaataataaaaatgatgatgatgattcgatgatgtggtgggaaggataaattaaa
ccgactttggggcagtgacaggcagtgtcagtgtcaaagacaaccatttgtagtcactatttctatcgaaggttgcaaattgaatggtggag
gagtatcaaaacgacacacatacttgaaaagatattttaataatataaaaaaattggtgatggcgtaataacaaacctagagctaattatta
tccttaatgataccaaatctatatgatacgatatttgttttaaaaagagtaaagactgacacttgagatgtgacactggcgatttcgctcac
gtcaccacttttcccacctcaaataacgcttacggctttatccattaattctaagtataattttaagtgtattttttcttgccaaattcaaa
tatatcttactaaatggatgaacattataaaattgttatcaaaaccattaaatgttcttataatttctttcgttcctccaatgtcatcccaa
gactttttgacctaatatatgatatatctaacttgctttggaatcgtatgacatatatcttcaaatacatatttcgtatttttttttcacga
aaactaatttagaaagtagaaaaccagctattttaaagaaaataaagtgtgtttatatatattctaaaacaatgctataagaacataagacc
aagatatatacaatgttattttatatttattattaagcattaacattgaaattaaaaatattaaacatgtataccaaagtaatcaacattgt
agttattactactctctctgttcatttttgtttgattgtttagaaaaaacacacatattaagaaaacatattaaatattgattataaatgta
ttatttttaatgttttacagttttctataactttaaaccaatgataatta;

### Selectable Markers

Genetic constructs may encode a selectable marker to enable selection of transformation events. There are many methods that have been described for the selection of transformed plants [for review see (Miki et al., Journal of Biotechnology, 2004, 107, 193-232) and references incorporated within]. Selectable marker genes that have been used extensively in plants include the neomycin phosphotransferase gene nptII (U.S. Pat. Nos. 5,034,322, 5,530,196), hygromycin resistance gene (U.S. Pat. No. 5,668,298), the bar gene encoding resistance to phosphinothricin (U.S. Pat. No. 5,276,268), the expression of aminoglycoside 3″-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. Nos. 5,463,175; 7,045,684). Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson et al., Nat Biotechnol, 2004, 22, 455-8). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of transgenic plants. Methods for positive selection using sorbitol dehydrogenase to convert sorbitol to fructose for plant growth have also been described (WO 2010/102293). Screenable marker genes include the beta-glucuronidase gene (Jefferson et al., 1987, EMBO J. 6: 3901-3907; U.S. Pat. No. 5,268,463) and native or modified green fluorescent protein gene (Cubitt et al., 1995, Trends Biochem. Sci. 20: 448-455; Pan et al., 1996, Plant Physiol. 112: 893-900).

Transformation events can also be selected through visualization of fluorescent proteins such as the fluorescent proteins from the nonbioluminescent Anthozoa species which include DsRed, a red fluorescent protein from the Discosoma genus of coral (Matz et al. (1999), Nat Biotechnol 17: 969-73). An improved version of the DsRed protein has been developed (Bevis and Glick (2002), Nat Biotech 20: 83-87) for reducing aggregation of the protein. Visual selection can also be performed with the yellow fluorescent proteins (YFP) including the variant with accelerated maturation of the signal (Nagai, T. et al. (2002), Nat Biotech 20: 87-90), the blue fluorescent protein, the cyan fluorescent protein, and the green fluorescent protein (Sheen et al. (1995), Plant J 8: 777-84; Davis and Vierstra (1998), Plant Molecular Biology 36: 521-528). A summary of fluorescent proteins can be found in Tzfira et al. (2005), Plant Molecular Biology 57: 503-516) and Verkhusha, et al. (2004), Nat Biotech 22: 289-296) whose references are incorporated in entirety. Improved versions of many of the fluorescent proteins have been made for various applications. Use of the improved versions of these proteins or the use of combinations of these proteins for selection of transformants will be obvious to those skilled in the art. It is also practical to simply analyze progeny from transformation events for the presence of the PHB thereby avoiding the use of any selectable marker.

### C. Modified Plants/Plant Parts

Recombinant/transgenic plant and plant parts are disclosed in which the disclosed constructs have been introduced. A transgenic plant includes, for example, a plant that comprises within its genome an exogenous polynucleotide introduced by a transformation step. The exogenous polynucleotide can be stably integrated within the genome such that the polynucleotide is passed on to successive generations. The exogenous polynucleotide may be integrated into the genome alone or as part of a recombinant DNA construct. A transgenic plant can also comprise more than one heterologous polynucleotide within its genome. Each exogenous polynucleotide may confer a different trait to the transgenic plant. A heterologous polynucleotide can include a sequence that originates from a foreign species, or, if from the same species, can be substantially modified from its native form.

### Genes of Interest Used to Transform Plants

Examples of genes/polynucleotides that can be included in the disclosed constructions include a CAS endonuclease such as CAS9 and one or more guide polynucleotide sequences, for example, single guide RNA (sgRNA), which target modification/deletion of a gene of interest. A Cas endonuclease is a Cas protein encoded by a Cas gene, and is capable of introducing a double strand break into a DNA target sequence that is endogenous to the plant. The Cas endonuclease is guided by a guide polynucleotide to recognize and optionally introduce a double/single strand break at a specific target site into the genome of a cell. The Cas endonuclease unwinds the DNA duplex in close proximity of the genomic target site and cleaves both DNA strands upon recognition of a target sequence by a guide polynucleotide, but only if the correct Protospacer-Adjacent Motif (PAM) is approximately oriented at the 3′ end of the target sequence.

The guide polynucleotide is any polynucleotide sequence having sufficient complementarity with a target polynucleotide sequence to hybridize with the target sequence and direct sequence-specific binding of a CRISPR complex to the target sequence. The degree of complementarity between a guide polynucleotide and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). A guide polynucleotide can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. A guide polynucleotide can be less than about 75, 50, 45, 40, 35, 30, 25, 20, 15, 12, or fewer nucleotides in length. The ability of a guide polynucleotide to direct sequence-specific binding of a CRISPR complex to a target sequence may be assessed by any suitable assay. For example, the components of a CRISPR system sufficient to form a CRISPR complex, including the guide polynucleotide to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide polynucleotide to be tested and a control guide polynucleotide different from the test guide polynucleotide, and comparing binding or rate of cleavage at the target sequence between the test and control guide polynucleotide reactions. Other assays are possible, and will occur to those skilled in the art.

An advantageous Cas endonuclease gene for use in the methods and systems of the present disclosure is a Cas9 endonuclease. However, other Cas endonucleases can be used, such as Cas12a, for example, LbCas12a (Wolter, et al., The Plant J. 100(5):1083-1094 (2019). The Cas endonuclease gene can be a plant codon optimized Streptococcus pyogenes Cas9 gene encoding a Cas9 endonuclease that can recognize any genomic sequence of the form N(12-30)NGG. The disclosed constructs (for example, constructs made using T-DNA) encodes one or multiple single guide RNAs (sgRNAs) and an endonuclease (e.g., Cas9), which together induce targeted DNA double-stranded breaks (DSBs). In eukaryotes, DSBs are primarily repaired through the error-prone non-homologous end joining (NHEJ) pathway, often resulting in small indels that disrupt the reading frame of a gene and abolish its function (i.e., targeted mutagenesis). If the T-DNA also contains a DNA sequence homologous to the locus targeted by a Cas9-sgRNA complex (i.e., DNA repair template), the DSB can be fixed via homologous recombination and precise modifications can be for gene targeting.

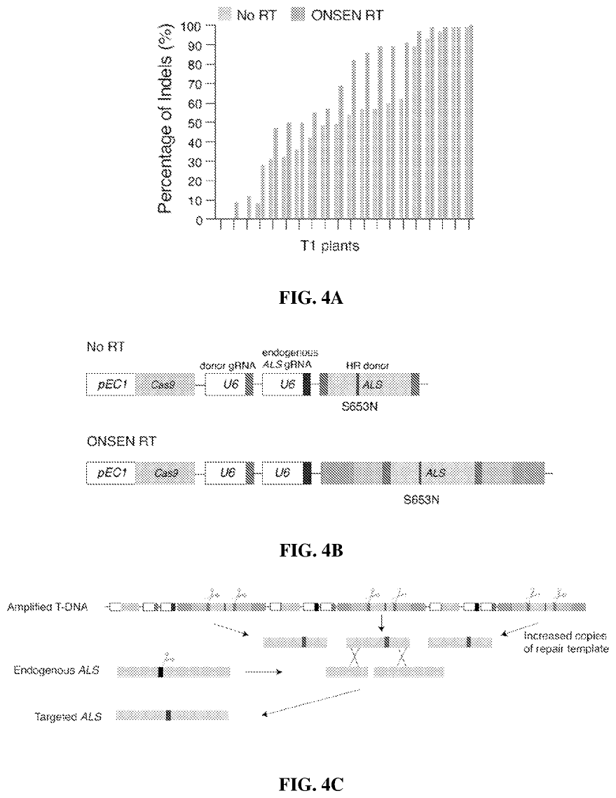

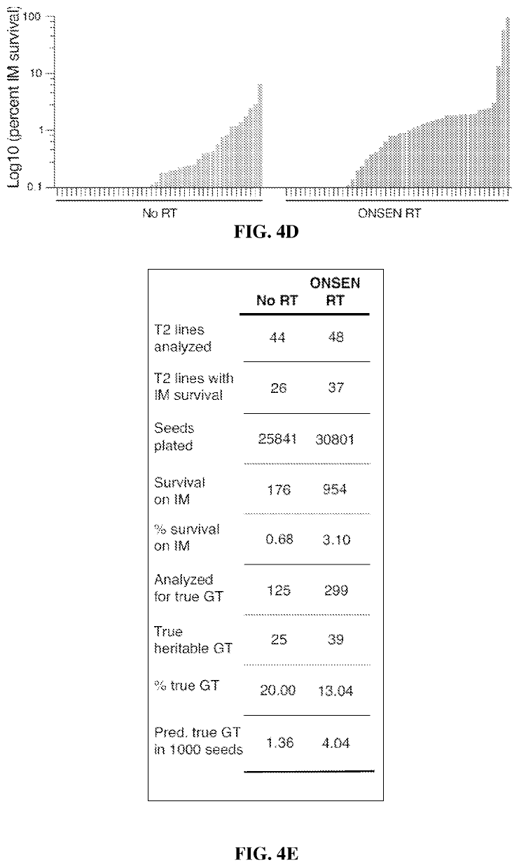

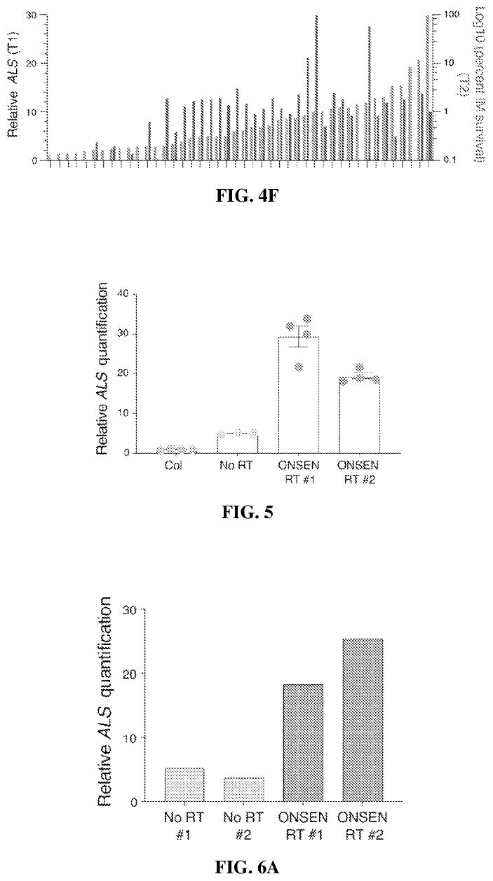

In some aspects a Cas endonuclease construct includes a Cas endonuclease expression cassette, two sgRNA expression cassettes and an expression cassette containing a nucleic acid which includes expression cassettes containing multiple pairs of DNA repeats and the donor nucleic acid (gene of interest) for homologous recombination (HR) (FIG. 4B). One sgRNA is programmed to cleave in the endogenous gene of interest, the other targets a sequence flanking the HR (homologous recombination) donor. Three DSBs are induced, leading to simultaneous activation of the target site and excision of the HR donor, which can then can be used as template for repair of the target site by HR²⁵.

Other genes of interest include, but are not limited to, a nucleic acid molecule which encodes a polypeptide, which nucleic acid molecule is able to confer a resistance to a disease causing plant parasite/pathogen. Late blight resistance genes are disclosed for example in U.S. Pat. No. 11,041,166 (nucleic acid molecules for resistance (R) genes that are capable of conferring to a plant, particularly a solanaceous plant (Solanaceae family), resistance to at least one race of a Phytophthora species (sp.) that is known to cause a plant disease in the plant. Two interesting examples of plant proteins that confer disease resistance in various crops in a dominant fashion are plant ferredoxin-like protein (PFLP) and hypersensitive response-assisting protein (HRAP).

Transgenic can include any cell, cell line, callus, tissue, plant part or plant, the genotype of which has been altered by the presence of exogenous nucleic acid including those transgenics initially so altered as well as those created by sexual crosses or asexual propagation from the initial transgenic. The alterations of the genome (chromosomal or extra-chromosomal) by conventional plant breeding methods, that does not result in an insertion of a foreign polynucleotide, or by naturally occurring events such as random cross-fertilization, non-recombinant viral infection, non-recombinant bacterial transformation, non-recombinant transposition, or spontaneous mutation are not intended to be regarded as transgenic.

The construct/nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the exogenous nucleic acid molecule.

In one embodiment the disclosed transgenic plant cell, cell line, callus, tissue, plant part or plant (or progeny thereof) includes more than one copy of the gene of interest integrated in its genome. The more than one copy of the gene of interest are present as concatemers (i.e., in a chain or series) in the genome of the transgenic plant cell, cell line, callus, tissue, plant part or plant.

In another embodiment, T-DNA amplification can be minimized to a single chromosomal copy, by transforming plants where specific DNA repair pathway proteins (for example, MRE11, ATR and RAD17, as shown in our study) have been inactivated. This embodiment minimizes genomic disruption of plant germplasm. Thus, the disclosed transgenic plant cell, cell line, callus, tissue, plant part or plant (or progeny thereof) includes one copy of the gene of interest integrated in its genome.

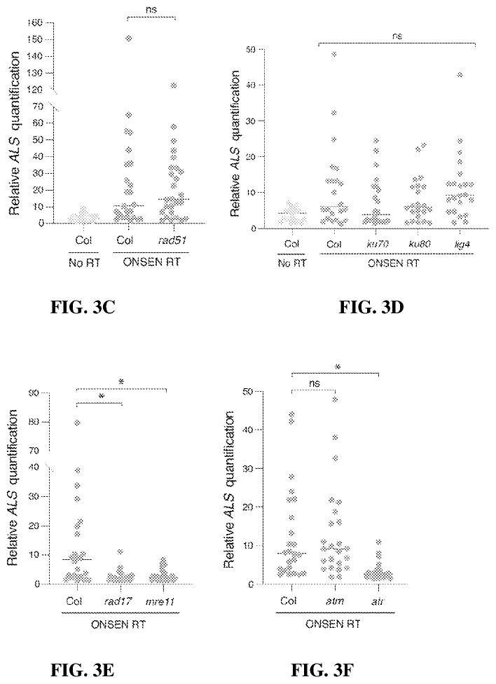

The data in this application demonstrates the ability to regulate T-DNA copy number both up (using DNA repeats) and down (by inactivating specific DNA repair genes). Regarding regulating downward T-DNA copy number, the data (FIGS. 3E and 3F), demonstrate that Agrobacterium-mediated plant transformation into three separate DNA repair mutants (in which rad17, mnre11, and atr are mutated to be nonfunctional) attenuates the T-DNA copy number increase, and even decreases it to a lower level than what is observed when wild-type plants are transformed with a standard vector (not containing any DNA repeats). Thus transforming into plants lacking these DNA repair proteins (either via mutation or by treating plants with a chemical inhibitor specifically targeting these DNA repair proteins) result in plants with a decreased T-DNA copy number.

In some forms a transgenic plant cell, cell line, callus, tissue, plant part or plant includes more than 5 fold, 6, 7, 8, 9, 101, 11, 12, 13, 14, 15, 16, 17, 18, 19, 20, 20, 22, 23, 24, 25, 26, 27, 28, 29, 30, 21, 32, 33, 34, 35, 36, 37, 38, 39, 40, 41, 42, 43, 44, 45, 46, 47, 48, 49, 50 and up to 60 fold increase in copy number of the inserted gene of interest, compared to the same plant cell, cell line, callus, tissue, plant part or plant transformed without the use of a construct including the gene of interest, and containing multiple pairs of DNA repeats.

In some forms, a transgenic plant cell, cell line, callus, tissue, plant part or plant does not include “helper” RTs.

### Plant Types

Suitable plant families include but are not limited to, Alliaceae, Amaranthaceae, Amaryllidaceae, Apocynaceae, Asteraceae, Boraginaceae, Brassicaceae, Campanulaceae, Caryophyllaceae, Chenopodiaceae, Compositae, Cruciferae, Cucurbitaceae, Euphorbiaceae, Fabaceae, Gramineae, Hyacinthaceae, Labiatae, Leguminosae-Papilionoideae, Liliaceae, Linaceae, Malvaceae, Phytolaccaceae, Poaceae, Pinaceae, Rosaceae, Scrophulariaceae, Solanaceae, Tropaeolaceae, Umbelliferae and Violaceae. Such plimts include, but are not limited to, Allium cepa, Amaranthus caudatus, Amaranthus retroflexus, Antirrhinum majus, Arabidopsis thaliana, Arachis hypogaea, Artemisia sp., Avena Sativa, Bellis perennis, Beta vulgaris, Brassica campestris, Brassica campestris ssp, Napus, Brassica campestris ssp, Pekinensis, Brassica juncea, Calendula officialis, Capsella bursa-pastoris, Capsicum annuum, Catharanthus roseus, Chemanthus cheiri, Chenopodium album, Chenopodium amaranticolor, Chenopodium foetidum, Chenopodium quinoa, Coriandrum sativum, Cucumis melo, Cucumis sativus, Glycine max, Gomphrena globosa, Gossypium hirsutum cv. Siv'on, Gypsophila elegans, Helianthus annuus, Hyacinthus, Hyoscyamus niger, Lactuca sativa, Lathyrus odoratus, Linum usitatissimum, Lobelia erinus, Lupinus nutabilis, Lycopersicon esculentum, Lycopersicon pimpinellifolium, Melilotus albus, Momordica balsamina, Myosotis sylvatica, Narcissus pseudonarcissus, Nicandra physalodes, Nicotiana benthamiana, Nicotiana clevelandii, Nicotiana glutinosa, Nicotiana rustica, Nicotiana sylvestris, Nicotiana tabacum, Nicotiana edwardsonii, Ocimum basilicum, Petunia hybrida, Phaseolus vulgaris, Phytolacca Americana, Pisum sativum, Raphanus sativus, Ricinius communis, Rosa sericea, Salvia splendens, Senecio vulgaris, Solanum lycopersicum, Solanum melongena, Solanum nigrum, Solanum tuberosum, Solanum pimpinellifolium, Spinacia oleracea, Stellaria media, Sweet Wormwood, Trifolium pratense, Trifolium repens, Tropaeolum majus, Tulipa, Vicia faba, Vicia villosa and Viola arvensis. Other plants that may be infected include Zea maize, Hordeum vulgare, Triticum aestivum, Oryza sativa and Oryza glaberrima.

The modified plan can be any commercially or scientifically valuable plant. For example, the plant can be a monocot, such as, maize, wheat, rice, sorghum, barley, oats, rice, soybean, peanut, pea, lentil, alfalfa, cotton, rapeseed, mums, lettuce, triticale, rye, pearl millet, finger millet, proso millet, foxtail millet, banana, bamboo, sugar cane, switchgrass, Miscanthus, asparagus, onion, garlic, chives, yam mums, arabidopsis, broccoli, cabbage, beet, quinoa, spinach, cucumber, squash, watermelon, beans, hibiscus, okra, apple, rose, strawberry, chile, garlic, sorghum, eggplant, eucalyptus, pine, a tree, an ornamental plant, a perennial grass and a forage crop, coniferous plants, moss, algae, etc. The plant can be a dicot. The term “dicot” as used herein refers to the subclass of angiosperm plants also knows as “dicotyledoneae” and includes reference to whole plants, plant organs (e.g., leaves, stems, roots, etc.), seeds, plant cells, and progeny of the same. Plant cell, as used herein includes, without limitation, seeds, suspension cultures, embryos, meristematic regions, callus tissue, leaves, roots, shoots, gametophytes, sporophytes, pollen, and microspores.

### III. Methods of Making and Using

The disclosed constructions and vectors are introduced into a plant of choice using methods known in the art. For example, DNA coding for the editing machinery (e.g., CAS9 and guide RNA) is introduced into plant callus, seed or embryonic tissue. Stably-transformed plants (events) are then recovered, optionally with the help of a selectable marker.

### A. Methods of Plant Transformation

Transformation protocols as well as protocols for introducing nucleotide sequences into plants may vary depending on the type of plant or plant cell targeted for transformation.

Suitable methods of introducing nucleotide sequences into plant cells and subsequent insertion into the plant genome include microinjection, electroporation, Agrobacterium-mediated transformation (Townsend et al., U.S. Pat. No. 5,563,055; Zhao et al. WO US98/01268), direct gene transfer (Paszkowski et al. (1984) EMBO J. 3:2717-2722), and ballistic particle acceleration (see, for example, Sanford et al., U.S. Pat. No. 4,945,050; Tomes et al. (1995) Plant Cell, Tissue, and Organ Culture: Fundamental Methods, ed. Gamborg and Phillips (Springer-Verlag, Berlin); and McCabe et al. Biotechnology 6:923-926 (1988)). A preferred method of is an agrobacterium mediated transformation, exemplified in the Examples of this application, the method of which is incorporated herein. The A. tumefaciens-mediated plant genetic transformation process requires the presence of two genetic components located on the bacterial Ti-plasmid. The first essential component is the T-DNA, defined by conserved 25-base pair imperfect repeats at the ends of the T-region called border sequences. The second is the virulence (vir) region, which is composed of at least seven major loci (virA, virB, virC, virD, virE, virF, and virG) encoding components of the bacterial protein machinery mediating T-DNA processing and transfer. The VirA and VirG proteins are two-component regulators that activate the expression of other vir genes on the Ti-plasmid. The VirB, VirC, VirD, VirE and perhaps VirF are involved in the processing, transfer, and integration of the T-DNA from A. tumefaciens into a plant cell (Hwang et al., 2017 doi.org/10.1199/tab.0186). This system has been extensively exploited in agarobacterium mediated plant transformations. One method used floral dip. In this method, transformation of female gametes is accomplished by simply dipping plant inflorescences for a few seconds into a 5% sucrose solution containing 0.01-0.05% (vol/vol) of the surfactant Silwet L-77. The optimal growth stage for transformation by floral dip was when plants contained numerous unopened floral buds. Treated plants are allowed to set seed which are then plated on a selective medium to screen for transformants. (described in detail in Clough et al., The Plant Journal 16(6):735-743 1998). The floral dip method is exemplified in the Examples section of this disclosure. Another method uses agroinfiltration. In the method a suspension of Agrobacterium tumefaciens is injected into a plant leaf, where it transfers the desired gene to plant cells. The first step of the protocol is to introduce a gene of interest to a strain of Agrobacterium. Subsequently the strain is grown in a liquid culture and the resulting bacteria are washed and suspended into a suitable buffer solution. This solution is then placed in a syringe (without a needle). The tip of the syringe is pressed against the underside of a leaf while simultaneously applying gentle counterpressure to the other side of the leaf. The Agrobacterium solution is then injected into the airspaces inside the leaf. Vacuum infiltration is another way to penetrate Agrobacterium deep into plant tissue. In this procedure, leaf disks, leaves, or whole plants are submerged in a beaker containing the solution, and the beaker is placed in a vacuum chamber. The vacuum is then applied, forcing air out of the stomata. When the vacuum is released, the pressure difference forces solution through the stomata and into the mesophyll.

Methods for transforming plant protoplasts are available including transformation using polyethylene glycol (PEG), electroporation, and calcium phosphate precipitation (see for example Potrykus et al., 1985, Mol. Gen. Genet., 199, 183-188; Potrykus et al., 1985, Plant Molecular Biology Reporter, 3, 117-128), Methods for plant regeneration from protoplasts have also been described [Evans et al., in Handbook of Plant Cell Culture, Vol 1, (Macmillan Publishing Co., New York, 1983); Vasil, IK in Cell Culture and Somatic Cell Genetics (Academic, Orlando, 1984)].

### B. Methods for Reproducing Transgenic Plants

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the transgene producing the desired level of desired polypeptide(s) in the desired tissue and cellular location.

In plastid transformation procedures, further rounds of regeneration of plants from explants of a transformed plant or tissue can be performed to increase the number of transgenic plastids such that the transformed plant reaches a state of homoplasmy (all plastids contain uniform plastomes containing transgene insert).

The cells that have been transformed may be grown into plants in accordance with conventional techniques. See, for example, McCormick et al. Plant Cell Reports 5:81-84(1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

The present invention will be further understood by reference to the following non-limiting examples.

### Examples

### Materials and Methods

### Plant Materials

Arabidopsis plants were grown under cool-white, fluorescent lights (~100 μmol m⁻² s⁻¹) in long-day condition (16-hour light/8-hour dark, 22° C.). The T-DNA insertion mutants atxr5/6 (At5g09790/At5g24330, SALK_130607/SAIL_240_HO1,²⁰), tsk/brul-4 (At3g18730, SALK 034207²⁷), rad51 (At5G20850, GK_134A0128), ku70-2 (AtIg16970, SALK_123114c²⁸), ku80-7 (At1g48050, SALK_112921²⁹), lig4-4 (At5g57160, SALK_044027³⁰), rad17-2 (At5g66130, SALK_009384³′), mre11 (At5g54260, SALK_028450³², atm (At3g48190, SALK_040423C³³), and atr (At5g40820, SALK_032841C³⁴) are in the Col-0 genetic background. They were obtained from the Arabidopsis Biological Resource Center (Columbus, Ohio).

### Cloning

All used in this study were derived from the DSB/DSB PcUbi4-2 and DSB/DSB AtEC1.1/1.2 vector as previously described¹¹. These vectors encode a Staphylococcus aureus CRISPR/Cas9 system. The derivative plasmids lacking the ubiquitin promoter and Cas9 sequence were made using the DSB/DSB PcUbi4-2 plasmid, which was digested using AscI and EcoRI, blunted with T4 DNA Polymerase (New England Biolabs, Ipswitch, MA), and re-ligated using T4 DNA Ligase (New England Biolabs, Ipswitch, MA). All retrotransposon (RT)-based plasmids (lacking Cas9) were generated by inserting an RT-ALS cassette (described below) in pace of ALS only cassette using the AatII and PacI restriction sites.

To make the ONSEN cassette, ONSEN (At1g11265, 4956 bp) was amplified from Co1-0 (−109 bp from the beginning of 5′LTR, to +114 bp from the end of the 3′LTR) and cloned into pCR2.1-TOPO (Invitrogen, Waltham, MA). An AscI site was then created in ONSEN at nucleotide position 987-994 (relative to 5′ LTR), replacing GTCACCGT (SEQ ID NO:3) with GGCGCGCC (SEQ ID NO:4). The ALS donor template (and the surrounding sgRNA binding sites) were amplified from DSB/DSB AtEC1.1/1.2 and inserted into the pCR2.1-TOPO-ONSEN vector using the AscI and BsrGI sites to generate pCR2.1-TOPO-ONSEN-ALS. The resulting ONSEN-ALS cassette was transferred to the binary plasmid (lacking Cas9) using the AatII and PacI restriction sites. For the binary plasmids expressing the other RTs, the LTRs and linker sequences of Copia13 (At2g13940), Copia2l (At5g44925), EVD (At5g17125) and GP3-1 (At3g11970) were mapped in the Arabidopsis genome and then synthesized at GenScript (Piscataway, NJ). The length of the 5′ linker (546 bp) and 3′ linker sequences (734 bp) synthesized for each RT was based on the length of the linker sequences in the ONSEN RT vector. A multicloning sequence that includes a NotI restriction site was inserted between the 5′ and 3′ linker sequences of each RT. The ALS donor template in DSB/DSB AtEC1.1/1.2 was removed from the vector using NotI and inserted in the NotI cloning site of the four synthesized RTs. Finally, the RT-ALS cassettes were transferred to the binary plasmid (lacking Cas9) using AatII and PacI.

The series of truncated ONSEN RT plasmids were created from a synthesized (GenScript) ONSEN-ALS cassette cloned into pUC57. The sequence of this synthesized ONSEN-ALS cassette is identical to the ONSEN-ALS cassette described in the previous paragraph, except for the insertion of restriction sites (each one cutting only at a single location) right before and after the different sections of ONSEN-ALS: 5′ LTR, 5′ linker, ALS, 3′ linker, and 3′ LTR. To remove a specific section of the synthesized ONSEN-ALS cassette, two restriction enzymes targeting the borders of that section were used to digest pUC57-ONSEN-ALS. The digested plasmid was then blunted using Quick Blunting kit (New England Biolabs, Ipswitch, MA) and re-ligated using T4 DNA Ligase (New England Biolabs, Ipswitch, MA) to generate the deletion. The random repeat sequence was generated using an online tool—Random DNA Sequence Generator online tool (The Maduro Lab, UC Riverside), synthesized at GenScript, and cloned into pUC57-ONSEN-ALS after removing the 5′ and 3′ LTRs. Finally, all modified ONSEN-ALS cassettes were cloned into the binary plasmid (lacking Cas9) using AatII and PacI. The binary plasmid lacking one of the two sgRNA genes was generated by digesting the No RT plasmid with XmaI and PacI, then blunting (Quick Blunting kit, New England Biolabs, Ipswitch, MA) and re-ligating using T4 DNA Ligase (New England Biolabs, Ipswitch, MA).

The binary plasmids (No RT and ONSEN RT) used for transformation into the Arabidopsis mutant backgrounds were modified to replace the kanamycin resistance gene (NPTII) with an hygromycin resistance gene. Briefly, the NPTII gene cassette was removed from the No RT and ONSEN RT plasmids by restriction digest. In parallel, the NPTII gene cassette (including promoter and terminator) was cloned into pAGM1311, and then modify using HiFi DNA Assembly Cloning Kit (New England Biolabs, Ipswitch, MA) to replace the complete coding sequence of NPTII with the coding sequence of the hygromycin resistance gene from pMDC7³⁴. The modified cassette was then reinserted into the No RT and ONSEN RT plasmids.

For the gene editing experiments involving the detection of mutations at the CRY2 locus, the DSB/DSB PcUbi4-2 plasmid was first modified by replacing the original ALS cassette with an ALS cassette lacking the sgRNA binding sites using AatII and PacI. The sgRNA binding sites were eliminated to prevent cutting of the chromosomal T-DNA locus by the Cas9-sgRNA complex, which could affect T-DNA quantification. To build the equivalent ONSEN RT plasmid, an ALS cassette without the sgRNA binding sites was inserted in the pCR2.1-TOPO-ONSEN vector using the AscI and BsrGI sites, and the resulting ONSEN-ALS cassette was then cloned into the DSB/DSB PcUbi4-2 plasmid at the AatII and Pact sites. Finally, the sgRNA gene targeting the ALS endogenous locus was replaced by a sgRNA gene targeting CRY2. This was done by digesting the ONSEN RT plasmid with XmaI and Pact and subcloning the fragment (containing the endogenous ALS gRNA gene) into a pENTR/D-Topo vector (Thermo fisher Scientific, Waltham, MA). The resulting plasmid was PCR amplified with a primer pair to change the ALS sgRNA spacer sequence to one of three different CRY2 sequences (sgRNA #1, 5′-AAGATCGCTGAAATCGTGTT-3′ (SEQ ID NO:5); sgRNA #2, 5′-GCAGGACCGGTTATCCGTTG-3′ (SEQ ID NO:6); and sgRNA #3, 5′-CCGATCATGATCTGTGCTTC-3′ (SEQ ID NO:7)). The amplified PCR products were then ligated with T4 DNA Ligase (NEB) and the CRY2 sgRNA genes were subcloned into the modified DSB/DSB PcUbi4-2 plasmids (i.e., lacking sgRNA binding sites) with XmaI and Pact.

To produce the ONSEN RT plasmid for gene targeting, the ONSEN cassette (containing ALS and the sgRNA binding sites) was amplified from the pCR2.1-TOPO-ONSEN-ALS vector and inserted into DSB/DSB AtEC1.1/1.2 using the AatII and Pac sites, after removing the ALS only cassette. The original DSB/DSB AtEC1.1/1.2 plasmid (No RT plasmid) served as a control.

### Plant Transformation

### Arabidopsis Transformation

Arabidopsis plants were transformed by using the floral dip method³⁵. Briefly, one day prior to floral dip transformation, 300 μL of a stationary Agrobacterium (strain GV310) liquid culture was used to inoculate 200 mL of Luria-Bertani (LB) containing 100 mg/L gentamycin, 100 mg/L spectinomycin and 50 mg/L rifamycin. The culture was incubated with shaking overnight at 28° C. The bacterial culture was spun down at 3,220×g for 25 minutes and resuspended in 200 mL of transformation solution (5% sucrose and 0.02% Silwet L-77). Arabidopsis flowers were dipped into the bacterial solution, gently agitated for 10 seconds, then stored horizontally in a tray with a blackout lid overnight in a long-day growth chamber. T1 plants were selected on ½ MS plates containing 1% sucrose, carbenicillin (200 μg/mL) and either kanamycin (100 μg/mL) or hygromycin (25 μg/mL). Herbicide-resistant seedlings were transferred to soil after 7-10 days on plates.

### Tobacco Transformation

Leaf pieces of in vitro-grown plants of Nicotiana tobaccum ‘Glurk’ were used as initial explant for particle bombardment. The procedure was performed with a Biolistic PDS-1000/He particle delivery system (Bio-Rad) using a protocol previously described45. In summary, 0.6 μm gold microcarriers (Bio-Rad) were defrosted and water-bath sonicated for 1 min. Plasmid DNA (final concentration of 1 μg μl-1) was added to the gold microcarriers and vortexed for 1 min. Calcium chloride (50 μl, 2.5 M) and 20 μl of 0.1 M spermidine were then added onto the inside of the cap of the tube and mixed by pipetting up and down 2-3 times. The cap of the tube was closed and the tube was tapped down to let all the components mix at the bottom. The mixture was centrifuged for 5 s at top speed and the supernatant was removed. The pellet was resuspended in 150 μl 100% ethanol vortexed for 1 min. The components were centrifuged for 5 s at top speed and the supernatant was removed. The pellet was then resuspended in 40 μl 100% ethanol. The macrocarriers were loaded on the macrocarrier holders. The gold-plasmid complex in the 100% ethanol solution was slowly loaded onto the center of the macrocarrier. The gold-plasmid DNA complex was then allowed to air dry on the macrocarriers for 5-10 min. The settings used for particle delivery were as follows: 2.5 cm distance gap between the rupture disk and macrocarrier, 9 cm target distance between the stopping screen and the target plate, 0.8 cm distance between the macrocarrier and the stopping screen, 28-29″ Hg vacuum, 5.0 vacuum flow rate and 4.5 vacuum vent rate.

Leaf pieces of tobacco plants were placed on tobacco pre-culture media, as previously described46, containing 4.3 g 1-1 of Murashige and Skoog (MS) salts, 1 ml 1-1 of Gamborg's vitamins 1000×, 1 ml 1-1 of Benzylaminopurine (1 mg ml-1), 1 ml 1-1 of napthalene acetic acid (1 mg ml-1), 1 ml 1-1 of p-chloro-phenoxy acetic acid (8 mg ml-1), 30 g 1-1 of sucrose and 6.5 g 1-1 of Difco agar, with pH adjusted to 5.7. Plates containing the leaf pieces were bombarded, removed from the delivery system, wrapped with PVC film and placed under light at 25° C. for 4 d. Leaf pieces were then transferred to selection/regeneration media containing 4.3 g 1-1 of MS salts. 1 ml 1⁻¹ of Gamborg's vitamins 1000×1 ml 1⁻¹ of benzylaminopurine (1 mg ml⁻¹), 0.1 ml 1⁻¹ of napthalene acetic acid (1 mg ml⁻¹), 30 g 1⁻¹ of sucrose, 75 mg 1⁻¹ of kanamycin and 6.5 g 1-1 of Difco agar, with pH adjusted to 5.7. The plates were kept under light at 25° C. and subcultured every 2 weeks until shoot formation. All plants were rooted under selection media. Leaves were collected for genomic DNA extraction 1.5 months after regeneration (total timing since bombardment: 3 months).

### Plant DNA Extraction

Leaves from 2-week-old plants (unless otherwise indicated) were homogenized in 500 μL DNA extraction buffer (200 mM Tris-HCl pH 8.0, 250 mM NaCl, 25 mM EDTA, and 1% SDS) and 50 μL phenol:chloroform:isoamyl alcohol (25:24:1). Each sample was centrifuged for 7 minutes at 16,000×g, and 300 μL of the aqueous layer was transferred to a 1.5 mL tube containing 300 μL isopropanol. Samples were vortexed, incubated at room temperature for 5 minutes, and then spun down at 16,000×g for 10 minutes. The supernatant was removed, and the pellets were washed with 70% ethanol. After centrifugation at 16,000×g for 5 minutes, the ethanol was removed and the pellets were dissolved in 100 μL of water.

### Agrobacterium DNA Extraction

GV310-transformed colonies were grown in liquid culture overnight at 28° C. 300 μL of each culture was transferred to a 1.5 ml tube and centrifuged for 1 minute at 16,000×g. The resulting pellet was resuspended in 250 μL of a lysozyme solution (200 mM CaCl2 with 1% lysozyme). The samples were incubated at 42° C. for 5 minutes and 750 μL of 96% ethanol was added, followed by centrifugation for 10 minutes at 16,000×g. The supernatant was removed, and the pellet was air dried for 10 minutes and resuspended in 100 μL of water.

### DNA-qPCR

Real-time PCR was carried out using a CFX96 Real-Time PCR Detection System (Bio-Rad, Hercules, CA) with a KAPA SYBR FAST qPCR Master Mix (2×) Kit (Kapa Biosystems, Wilmington, MA). Relative quantities were determined by the 2(-delta Ct) method³⁶ using Actin7 (At5g09810) and the gene coding for a hypothetical protein found at the NCBI accession number: WP_046033610.1 (SEQ. ID NO:4) and the L25 gene as the normalizer for DNA extracted from Arabidopsis, Agrobacterium, and tobacco, respectively.

(SEQ ID NO: 35)
MKYIFLLSAFLIFLFSAFIYFFPIFGAVVCPPCFGFVEVEGGIYIDASLNADLEKDKILSN
LDAAHLLLREVYGEVEAPLPAIFLCVSKNCATYLGRRGEKASSFGHWAIVVYRDGN
NSGILAHELSHIEIGFRLGFYNMSSVPIWFDEGVAVVASQDRRYLNVDPSGRLSCKEG
VSGAVIADLDEWWRRASIGDVGIYSAAACEVMKWMDRRGNEGSLVRLLDLLRSGE
SFDVAF

Relative quantities of ALS in Arabidopsis plants were calculated by using Co1-0 DNA as the calibrator. For relative quantification of pea3A(T) and NPTII in Arabidopsis, and of ALS in Agrobacterium, a DNA sample from a “No RT” transformant was used as the calibrator. Graphpad prism 9 was used to analyse the data. All primer sequences can be found in Table 1.

TABLE 1
List of primers used in this study.
Primers for DNA q-PCR

| Target | Forward primer | Reverse primer |
|---|---|---|
| Actin7 | TCGTGGTGGTGAGTTTGTTAC | CAGCATCATCACAAGCATCC (SEQ |
| | (SEQ ID NO: 9) | ID NO: 10) |
| ALS | GCTGGCTTTGCAAGGGATG (SEQ | CCATCAGTCAACTCATCAAGG (SEQ |
| | ID NO: 11) | ID NO: 12) |
| pea 3A | CCGGCTCGTATGTTGTGTGG (SEQ | CTGGTGTGTGGGCAATGAAACTG |
| | ID NO: 13) | (SEQ ID NO: 14) |
| NPTII cassette | GGTGGAGCACGACACTCTCG | GATGGCATTTGTAGGTGCCACC |
| | (SEQ ID NO: 15) | (SEQ ID NO: 16) |
| WP_046033610.1 | GCCTAGCCTAAAGCCAATCTC | GCGTGTCCAAGAATTGCGCC (SEQ |
| | (SEQ ID NO: 17) | ID NO: 18) |

Primers to assess editing events

| Target | Forward Primer | Reverse Primer |
|---|---|---|
| CRY2 guide #1 region | ATCCGTTGGTGGATGCCGGAAT | CACACATCTACATGTTAGGC (SEQ |
| | (SEQ ID NO: 19) | ID NO: 20) |
| CRY2 guide #2 region | GTTGTTAACTAGAGCTTGGTCTCC | CATGGATGATGGATCCATTCAGTTG |
| | (SEQ ID NO: 21) | (SEQ ID NO: 22) |
| CRY2 guide #3 region | CACAGTCTTTGATTCGAAGATC | GGTCCCGAACTAACGAAACAGG |
| | (SEQ ID NO: 23) | (SEQ ID NO: 24) |
| endogenous ALS | AGTACGTTGATGGGGCTGG (SEQ | CCAATGTCTGATTAGTGCTTCTGG |
| | ID NO: 25) | (SEQ ID NO: 26 |

### Fluorescence In Situ Probe Design

Primary FISH probes were designed as previously described with modifications³⁷. First, a pool of 30 nucleotides (nt) primary targeting sequences were designed with OligoArray2.1³⁸, with the following parameters: sequence length 30 nt; minimum melting temperature 66° C.; maximum melting temperature 100° C.; secondary structure melting temperature limit 76° C.; cross-hybridization melting temperature limit 72° C.; minimum GC content 30%; maximum GC content 90%; avoiding 6 or more consecutive A, T, G or C's; allowing at most 20-nt overlap between adjacent target sequences. The primary targeting sequences were blasted against the TAIR10 genome to ensure specificity. Then, a 30 nt secondary probe binding sequence (reverse complement of the dye-labeled secondary probe sequence) was appended to the 3′ end of each primary targeting sequence to generate the full-length primary probes (Integrated DNA Technologies, Coralville, IA). To detect ALS and the NPTII gene, 39 and 29 primary probes were designed, respectively (sequences shown below)

ALS probes
(SEQ ID NO: 27)
GAGAGATGAAACCGGTGATTATCAGAACCTGATCCGATTGGAACCGTCCCAAGCGTTGCG
TCATAACGGAAGGAGATGGCCGGATTAAATGATCCGATTGGAACCGTCCCAAGCGTTGCG
ATGTGATTTGTCCGCACCAAGAACATGTGTGATCCGATTGGAACCGTCCCAAGCGTTGCG
CAATGCTGGATACACCAGGACCTTACCTGTGATCCGATTGGAACCGTCCCAAGCGTTGCG
CAAAGAAAGCAGATCTCCGAGAAGCTATTCGATCCGATTGGAACCGTCCCAAGCGTTGCG
AGGACGAGATATTCCCGAACATGTTGCTGTGATCCGATTGGAACCGTCCCAAGCGTTGCG
GAGCTCACACATTTCTCGGGGATCCGGCTCGATCCGATTGGAACCGTCCCAAGCGTTGCG
GTTATGCAATGGGAAGATCGGTTCTACAAAGATCCGATTGGAACCGTCCCAAGCGTTGCG
GTACTTTTATTAAACAACCAGCATCTTGGCGATCCGATTGGAACCGTCCCAAGCGTTGCG
CTAGCCACTATTCGTGTAGAGAATCTTCCAGATCCGATTGGAACCGTCCCAAGCGTTGCG
GGAGATGGAAGCTTTATAATGAATGTGCAAGATCCGATTGGAACCGTCCCAAGCGTTGCG
GCTAACCCTGATGCGATAGTTGTGGATATTGATCCGATTGGAACCGTCCCAAGCGTTGCG
TTTGGACTTCCTGCTGCGATTGGAGCGTCTGATCCGATTGGAACCGTCCCAAGCGTTGCG
TGGCTATCATCAGGAGGCCTTGGAGCTATGGATCCGATTGGAACCGTCCCAAGCGTTGCG
GCGCAGTTCTACAATTACAAGAAACCAAGGGATCCGATTGGAACCGTCCCAAGCGTTGCG
AGTACTGGTGTCGGGCAACATCAAATGTGGGATCCGATTGGAACCGTCCCAAGCGTTGCG
CTTGATGAGTTGACTGATGGAAAAGCCATAGATCCGATTGGAACCGTCCCAAGCGTTGCG
GAAGCTATTCCTCCACAGTATGCGATTAAGGATCCGATTGGAACCGTCCCAAGCGTTGCG
CAGAAGTTTCCGTTGAGCTTTAAGACGTTTGATCCGATTGGAACCGTCCCAAGCGTTGCG
GGAGTTTGGAGGAATGAGTTGAACGTACAGGATCCGATTGGAACCGTCCCAAGCGTTGCG
GAGAACCGAGCGGAGGAGCTTAAGCTTGATGATCCGATTGGAACCGTCCCAAGCGTTGCG
AAGCTGGCTTTGCAAGGGATGAATAAGGTTGATCCGATTGGAACCGTCCCAAGCGTTGCG
AAGACTCCTCATGTGTCTGTGTGTGGTGATGATCCGATTGGAACCGTCCCAAGCGTTGCG
ATTGATATTGACTCGGCTGAGATTGGGAAGGATCCGATTGGAACCGTCCCAAGCGTTGCG
GAGGCTTTTGCTAGTAGGGCTAAGATTGTTGATCCGATTGGAACCGTCCCAAGCGTTGCG
GTAAGGTTTGATGATCGTGTCACGGGTAAGGATCCGATTGGAACCGTCCCAAGCGTTGCG
TACGCTGTGGAGCATAGTGATTTGTTGTTGGATCCGATTGGAACCGTCCCAAGCGTTGCG
CTTTTGGTTCATTTTAACCTTCTGTAAACAGATCCGATTGGAACCGTCCCAAGCGTTGCG
GAAATAGGGTAATTCAAAATCTAGCTTGATGATCCGATTGGAACCGTCCCAAGCGTTGCG
CTGTGTCCACATTATCAGTTTTGTGTATACGATCCGATTGGAACCGTCCCAAGCGTTGCG
TAAAACTACGATGTCATCGAGAAGTAAAATGATCCGATTGGAACCGTCCCAAGCGTTGCG
TGTGCTCATATGGGCCGTGGTTTCCAAATTGATCCGATTGGAACCGTCCCAAGCGTTGCG
GAAAATGCTCTTACCATTGGTTTTTAATTGGATCCGATTGGAACCGTCCCAAGCGTTGCG
GTCACTGGGTTAATATCTCTCGAATCTTGCGATCCGATTGGAACCGTCCCAAGCGTTGCG
AATTCGTTATTAGGGTTCTAAGCTGTTTTAGATCCGATTGGAACCGTCCCAAGCGTTGCG
ATTGCGAAATGCGAATGGTAAATTGAGTAAGATCCGATTGGAACCGTCCCAAGCGTTGCG
CGGTTTACTCCTTGTGACTGGCTCAGTTTGGATCCGATTGGAACCGTCCCAAGCGTTGCG
CTGCTTTTTGGTTTACGTCAGACTACTACTGATCCGATTGGAACCGTCCCAAGCGTTGCG
GGTAATTTGAGTTTCTTTTAGTTGTTGATCGATCCGATTGGAACCGTCCCAAGCGTTGCG
NPTII probes
(SEQ ID NO: 28)
CAAGACCGACCTGTCCGGTGCCCTGAATGACCCATGATCGTCCGATCTGGTCGGATTTGT
ATGGACCCCCACCCACGAGGAGCATCGTGGCCCATGATCGTCCGATCTGGTCGGATTTGT
CGATGCCTGCTTGCCGAATATCATGGTGGACCCATGATCGTCCGATCTGGTCGGATTTGT
AAAACGTCCGCAATGTGTTATTAAGTTGTCCCCATGATCGTCCGATCTGGTCGGATTTGT
TCGGCGTTAATTCAGTACATTAAAAACGTCCCCATGATCGTCCGATCTGGTCGGATTTGT
CCCGAATTAATTCGGCGTTAATTCAGTACACCCATGATCGTCCGATCTGGTCGGATTTGT
CACCTAAAGTCCCTATAGATCCCCCGAATTCCCATGATCGTCCGATCTGGTCGGATTTGT
AAAATCCAGATCACCTAAAGTCCCTATAGACCCATGATCGTCCGATCTGGTCGGATTTGT
CCTAAAACCAAAATCCAGTACTAAAATCCACCCATGATCGTCCGATCTGGTCGGATTTGT
TCACGTGTTGAGCATATAAGAAACCCTTAGCCCATGATCGTCCGATCTGGTCGGATTTGT
TAGGGTTCTTATAGGGTTTCGCTCACGTGTCCCATGATCGTCCGATCTGGTCGGATTTGT
AGTAGTTCCCAGATAAGGGAATTAGGGTTCCCCATGATCGTCCGATCTGGTCGGATTTGT
AATAATGTGTGAGTAGTTCCCAGATAAGGGCCCATGATCGTCCGATCTGGTCGGATTTGT
GATCGACATCGAGTTTCTCCATAATAATGTCCCATGATCGTCCGATCTGGTCGGATTTGT
TAGCTAGAGTCGATCGACATCGAGTTTCTCCCCATGATCGTCCGATCTGGTCGGATTTGT
TCTGGGGTTCGGATCGATCCTCTAGCTAGACCCATGATCGTCCGATCTGGTCGGATTTGT
CTGAGCGGGACTCTGGGGTTCGGATCGATCCCCATGATCGTCCGATCTGGTCGGATTTGT
ATCGCCTTCTTGACGAGTTCTTCTGAGCGGCCCATGATCGTCCGATCTGGTCGGATTTGT
CATCGCCTTCTATCGCCTTCTTGACGAGTTCCCATGATCGTCCGATCTGGTCGGATTTGT
TCGCCGCTCCCGATTCGCAGCGCATCGCCTCCCATGATCGTCCGATCTGGTCGGATTTGT
GCTTTACGGTATCGCCGCTCCCGATTCGCACCCATGATCGTCCGATCTGGTCGGATTTGT
AATGGGCTGACCGCTTCCTCGTGCTTTACGCCCATGATCGTCCGATCTGGTCGGATTTGT
GCTTGGCGGCGAATGGGCTGACCGCTTCCTCCCATGATCGTCCGATCTGGTCGGATTTGT
CTACCCGTGATATTGCTGAAGAGCTTGGCGCCCATGATCGTCCGATCTGGTCGGATTTGT
CATAGCGTTGGCTACCCGTGATATTGCTGACCCATGATCGTCCGATCTGGTCGGATTTGT
GTGTGGCGGACCGCTATCAGGACATAGCGTCCCATGATCGTCCGATCTGGTCGGATTTGT
TGGCCGGCTGGGTGTGGCGGACCGCTATCACCCATGATCGTCCGATCTGGTCGGATTTGT
GCTTTTCTGGATTCATCGACTGTGGCCGGCCCCATGATCGTCCGATCTGGTCGGATTTGT
AATATCATGGTGGAAAATGGCCGCTTTTCTCCCATGATCGTCCGATCTGGTCGGATTTGT

The secondary probe sequences were adapted from a previous report³⁷.

The secondary probes for ALS and NPTII were conjugated to 5′ Alex Fluor 488 and ATTO 590, respectively (IDT).

Secondary probes
(SEQ ID NO: 29)
5' Alex Fluor 488-CGCAACGCTTGGGACGGTTCCAATCGGATC;
(SEQ ID NO: 30)
5' ATTO 590-ACAAATCCGACCAGATCGGACGATCATGGG.

### Fluorescence In Situ Hybridization

Leaves from 3-week-old plants were fixed in cold 4% formaldehyde in Tris buffer (10 mM Tris-HCl pH 7.5, 10 mM NaEDTA, 100 mM NaCl) for 20 minutes, and then washed twice in Tris buffer. The leaves were chopped with a razor blade in 500 μl LB01 buffer (15 mM Tris-HCl pH7.5, 2 mM NaEDTA, 0.5 mM spermine-4HCl, 80 mM KCl, 20 mM NaCl and 0.1% Triton X-100), and the resulting slurry was filtered through a 30 μM mesh (Sysmex Partec, Gorlitz, Germany). The filtered solution was mixed 1:1 with sorting buffer (100 mM Tris-HCl pH 7.5, 50 mM KCl, 2 mM MgCl₂, 0.05% Tween-20 and 5% sucrose), spread onto a coverslip, and dried. Cold methanol was added to the coverslips for 3 minutes, followed by TBS-Tx (20 mM Tris pH 7.5, 100 mM NaCl, 0.1% Triton X-100). 0.1 mg/ml of RNase A in 2×SSC (0.3M NaCl, 30 nM sodium citrate, pH 7.0) was added onto the coverslips and incubated at 37° C. for 45 minutes, followed by two washes in 2×SSC. Pre-hybridization buffer (2×SSC, 50% Formamide and 0.1% Tween 20) was added for 30 minutes at room temperature. Probes were added to the hybridization buffer at a concentration of 1 μM, added to each coverslip and incubated at 80° C. for 3 minutes, followed by an overnight 37° C. incubation in a humid chamber. Coverslips were washed twice with SSC-0.1% Tween at 60° C. for 15 minutes, and once for 15 minutes at room temperature. Secondary probes (Alexa-488 or Alexa-595) at a concentration of 40 nM in buffer (2×SSC+40% formamide) were added to coverslips. The coverslips were incubated at room temperature for 30 minutes, washed with secondary wash buffer (2×SSC+40% formamide), and washed twice with 2×SSC. Coverslips were mounted on microscope slides with VectaShield containing DAPI (Vector Laboratories, Burlingame, CA). Nuclei were imaged under a Nikon Eclipse Ni-E microscope with a 100× CFI PlanApo Lamda objective (Nikon, Minato City, Tokyo, Japan) and an Andor Clara camera. Z-series optical sections of each nucleus were obtained at 0.3 μm steps. Images were deconvolved by FIJI using the DeconvolutionLab plugin^{39,40} The nuclei selected for imaging were ≤55 μm³ to enrich for 2C nuclei⁴¹. The nuclear volume was measured using FIJI with the 3D ImageJ suite^{40,42}

### Library Construction, Sequencing and Bioinformatic Analyses

DNA sequencing libraries were prepared at the Yale Center for Genome Analysis. Genomic DNA was sonicated to a mean fragment size of 350 bp using a Covaris E220 instrument (Covaris, Woburn, MA) and libraries were generated using the xGen Prism library prep kit for NGS (Integrated DNA Technologies, Coralville, IA). Paired-end 150 bp sequencing was performed on an Illumina NovaSeq 6000 using the S4 XP workflow (Illumina, San Diego, CA). Raw FASTQ files were pre-processed and trimmed using the fastp tool⁴³(—length_required 20—average_qual 20—detect_adapter_for_pe-w 10). Subsequently, the command line program grep (Free Software Foundation, Boston, MA) was used to search FASTQ files using the 20 bp sequences inside the T-DNA neighboring the left border (LB) and right border (RB) sites (and their reverse complement) as a query. The filtered sequences were then processed using BioPython⁴⁵ to isolate flanking sequence tags (FSTs) adjacent to the LB and RB. FSTs were then used as BLAST queries⁴⁶ to identify regions of the genome where T-DNA sequences were inserted. Further supporting reads were obtained by mapping the FASTQ files to the genome (TAIR10⁴⁷) using BWA-MEM⁴³ and isolating reads for which only a single read of a mate pair maps to the genome at the identified point of insertion, as the other will read maps to the T-DNA insertion. Assembling the T-DNA insertion junctions was then done using the MAFFT multiple sequence aligner⁴⁹ and a reference sequence was manually created. Insert sites were confirmed using PCR and Sanger sequencing.

Estimation of transgene sequence copy number was achieved by mapping reads to a panel of 7,535 genes, along with three regions of the T-DNA transgene. The selected genes are reported in the PLAZA 5.0 database⁴⁰ to originate from single gene families in the genome (omitting plastid genes and At3g48560 from which the ALS sequence of the transgene derives). The read counts from these alignments were extracted using SAMtools idxstats function⁵¹ and normalized to Bins Per Million mapped reads (BPM; [Reads Per Kilobases]/[sum(Reads Per Kilobases)*1\hat{ }e6]). The mean BPM of the selected genes were taken as the normalized copy number of a single copy gene in the genome and the relative fold change, in BPM, for regions of the T-DNA transgene was calculated using this as a reference.

Measurement of coverage around mapped T-DNA insertion sites was done using the bamCompare function of the deepTools package⁵² which scales by read count and returns the log 2 ratio of two alignments for a genome split into equally sized bins. The bin size was set to 20 and the genome alignment of each line was compared with a wild-type Co1-0 sequenced at the same time.

Internal T-DNA junctions were assembled in a manner similar to the identification of insertion junctions with the genome but using reads with FSTs that match the binary vector used to transform the plants. Once assembled, the FASTQ files were searched again with consensus intersection sequences (±15 bp from either the point of intersection or edge of the filler sequence) and only intersections with two or more independent read pairs supporting it were retained.

### Gene Editing

To assess the efficiency of targeted mutagenesis of CRY2, genomic DNA was extracted from individual 2-week-old T1 plants and used to amplify the CRY2 gene. The resulting PCR products were sequenced and analyzed for INDEL frequency by Inference of CRISPR Edits (ICE) analysis (Synthego Performance Analysis, ICE Analysis. 2019. V3.0. Synthego).

To measure IPGT rates, T2 seeds from individual T1 lines were plated on ½ MS plates containing 5 mM Imazapyr (IM) and 1% sucrose. Seeds were stratified for 3 days at 5° C. The seed count for each plate was determined by using the ImageJ ‘analyze particles’ function (size 5-inf. pixels², 0-1 circularity) after binary processing. The seeds were allowed to germinate for one week in long day conditions and the imazapyr resistant seedling were then counted manually.

The true gene targeting events were characterized as previously described¹⁰, with some modifications. Briefly, the endogenous ALS locus was amplified with primers 11 and 12 (both outside of the donor template to prevent inclusion of ectopic events), and the PCR product was Sanger sequenced. The codon corresponding to amino acid 653 and the gRNA binding site were analyzed for a gene targeting event using Sequencher 5.4.6 (Gene Codes Corporation, Ann Arbor, MI). Samples with different types of editing events were re-analyzed using Synthego ICE or subcloning and further Sanger sequencing. Plants were considered as having undergone true gene targeting when samples displayed, at a minimum, ~50% GT-edited sequences.

### Statistics and Reproducibility

Statistical analyses were performed using GraphPad Prism v.9.4.0 for macOS (www.graphpad.com). The sample sizes, statistical tests and P values are indicated in the figure legends. All experiments in this study were performed at least three times with similar results.

### Results

As a strategy to increase T-DNA copy number in Arabidopsis, it was tested whether induction of transgene amplification may be accomplished by including retrotransposon (RT)-derived sequences within a T-DNA. To assess if RT-derived DNA sequences increase T-DNA copy number, a T-DNA vector was designed based on the ONSEN family of long terminal repeat (LTR) RTs^{9,10}. Part of the sequence coding for the gag and pol genes of an ONSEN RT (At1g11265) was first replaced with DNA from the Arabidopsis ACETOLACTATE SYNTHASE (ALS) locus. The aim of using this ALS fragment were: 1) to measure T-DNA copy number relative to the Arabidopsis genome and 2) to serve as a repair template in gene targeting assays (FIG. 1A). The resulting ONSEN RT was then subcloned into the T-DNA region of a binary vector modified from a previously described gene targeting Study¹¹. Identical plasmids, except for the presence of ONSEN sequences surrounding ALS, were then used to transform Arabidopsis Co1-0 plants via Agrobacterium using the floral dip method (FIG. 1B)¹². Genomic DNA was extracted from individual first-generation transformed (T1) plants and ALS quantification was performed by quantitative PCR using a primer set that can amplify both endogenous and T-DNA-associated ALS sequences. It was found that T1 plants transformed with the plasmid containing ONSEN sequence (ONSEN RT) had, on average, higher ALS copy numbers compared to untransformed plants or T1 plants transformed with the control plasmid lacking ONSEN (No RT) (FIG. 1C). Individual T1 plants transformed with ONSEN RT showed wide variation in ALS levels, with copy number sometimes reaching 50-fold increase compared to Col. In contrast, T1 plants transformed with the plasmid lacking ONSEN rarely showed more than 10-fold increase over Col (FIG. 1C). To determine if other parts of the T-DNA were being amplified, the relative levels of the Pea3A terminator and the kanamycin-resistance gene NPTII present at the 5′ and 3′ regions of the T-DNA, respectively, were quantified (FIG. 1B). Similar increases in copy numbers for ALS, pea3A(T), and the NPTII cassette were detected in individual T1 plants transformed with ONSEN RT (FIG. 1D), indicating that the entire T-DNA, and not solely ONSEN-embedded ALS, was amplified.

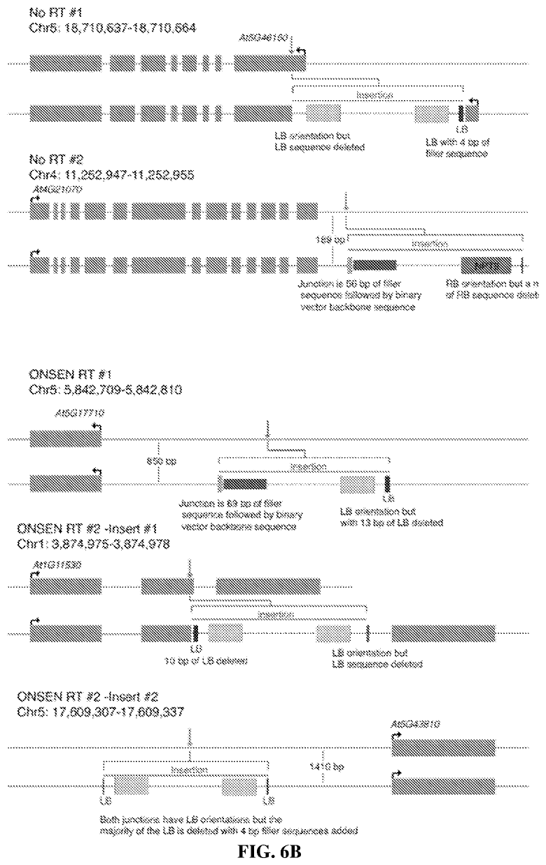

To characterize the structural organization of the ONSEN-driven T-DNA in the Arabidopsis genome (i.e., to determine whether the ONSEN-mediated T-DNA copy increase is due to concatenation or multiple independent insertions at different loci), DNA fluorescence in situ hybridization (FISH) experiments were conducted with probes designed to detect ALS and NPTII. In nuclei of T1 plants transformed with ONSEN RT and confirmed by DNA-qPCR to contain large numbers of T-DNA copies (>10 copies vs Col) (data not shown, and FIG. 5) bright and overlapping signals for ALS and NPTII were observed (data not shown shown). 1-2 One to two FISH signals per nuclei were typically detected. By contrast, FISH signals in nuclei from Col or T1 plants transformed using the plasmid lacking ONSEN were either undetectable or much weaker (data not shown, and FIG. 5). The fact that overlapping and high-intensity FISH signals can be observed for ALS and NPTII strongly suggests that ONSEN-induced T-DNA amplification results from concatenation rather than an increase in T-DNA insertion sites in the Arabidopsis genome (FIG. 11A-11C, with FIG. 11C depicting concatenation, in which no helper plasmid is used to provide RT replication proteins; compared to endogenous RT insertion into the genome (FIG. 11A, and ‘copy and paste” retrotransposon replication which requires a helper plasmid encoding the RT replication proteins, (FIG. 11B)). To validate this, the genome of a Col control and four T1 plants (two plants transformed with each type of T-DNA plasmid) were sequenced and characterized by different levels of T-DNA amplification (FIG. 6A). Analysis of the sequencing data confirmed the increase in copy number of the T-DNA and showed that these plants contained only one or two T-DNA insertion sites (FIGS. 1E-1F and FIG. 6B), thus confirming that T-DNA amplification is due to increased copies of T-DNA concatenation and not additional T-DNA insertion sites in the genome.

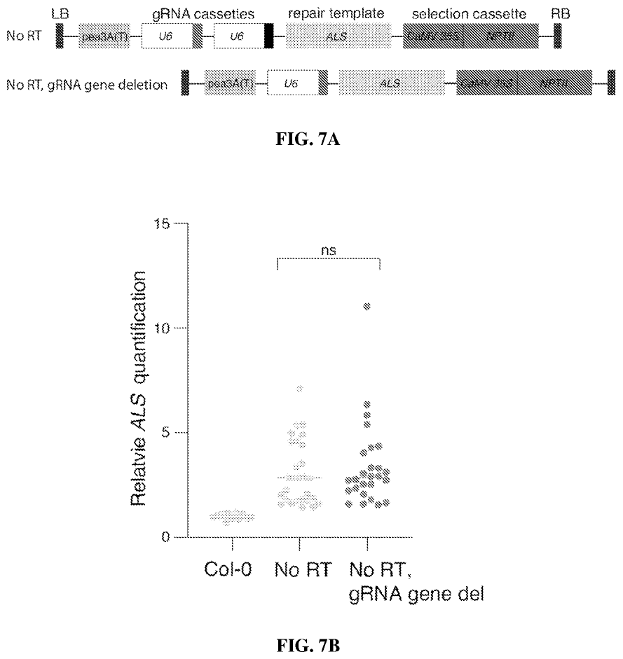

The mechanism(s) involved in ONSEN-mediated T-DNA amplification was investigated, next. Initial studies assessed if T-DNA amplification could be conferred by sequences from RTs other than ONSEN. Four different Arabidopsis LTR RTs (two Copia-type and two Gypsy-type) were tested by replacing part of their gag-pol sequence with the same ALS fragment present in ONSEN RT (FIG. 1A and FIG. 2A), and a similar effect of increased T-DNA copies compared to the ONEN RT plasmid was observed (FIG. 2B). The region(s) of the ONSEN RT involved in T-DNA amplification was then investigated by generating a series of binary plasmids containing various deletions of the ONSEN RT plasmid (FIG. 2C). The results show that the LTRs had the most impact on amplification (FIG. 2D). The hypothesis that the effect of the LTRs on T-DNA amplification is mainly caused by their repetitive nature was tested and confirmed by assessing T-DNA levels induced by a binary plasmid with random repeated sequences (RR) of the same length and GC content as the ONSEN LTRs (FIGS. 2E-2F). To validate this result, additional sets of artificial DNA repeats composed of different lengths (220 bp versus 440 bp) and GC content (26%, 55% and 73%) were tested. The results showed that all DNA repeats could increase T-DNA copy number, although high GC levels (73%) led to a lower increase (RR versus RR2, P<0.07; just outside of the statistical cut-off of P<0.05) (data not shown). Interestingly, not all DNA direct repeats within a T-DNA contribute to amplification, as removal of one of the two identical sgRNA genes, which eliminates a repeated Arabidopsis U6-26 promoter sequence of 387 bp, had no effect on T-DNA copy number (FIGS. 7A and 7B). Thus, multiple pairs of DNA repeats, and/or a linker sequence of defined length separating individual repeats, may be required to induce T-DNA amplification.

Additional studies tested whether DNA repeats also contribute to increasing transgene copy number in plants transformed using biolistic particle delivery (BPD). The results from BPD-transformed tobacco plants did not indicate any effect of the ONSEN repeats (data not shown), thus suggesting differences in the mechanisms involved in exogenous DNA integration.

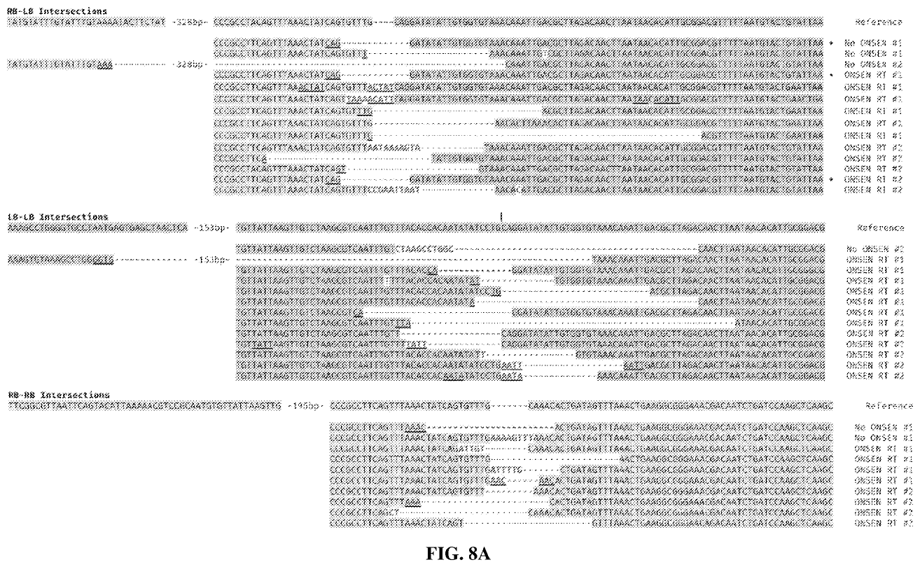

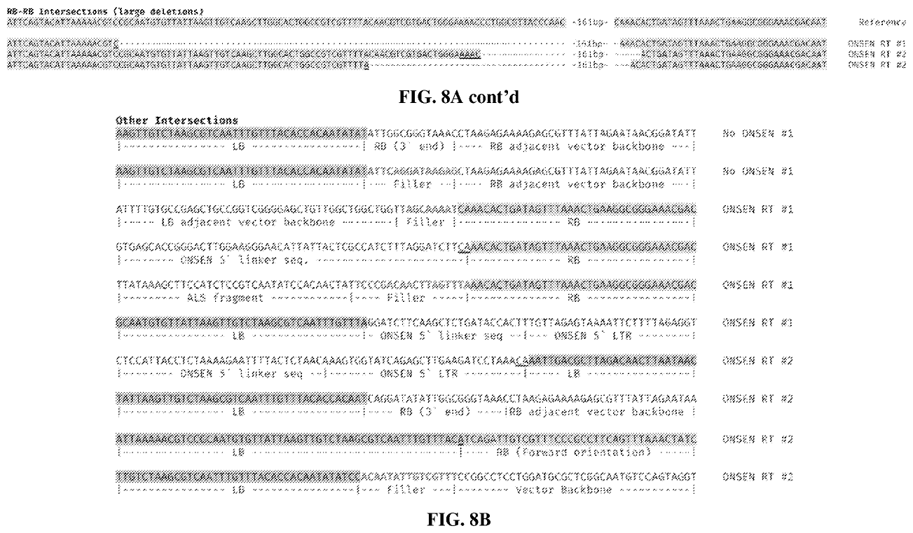

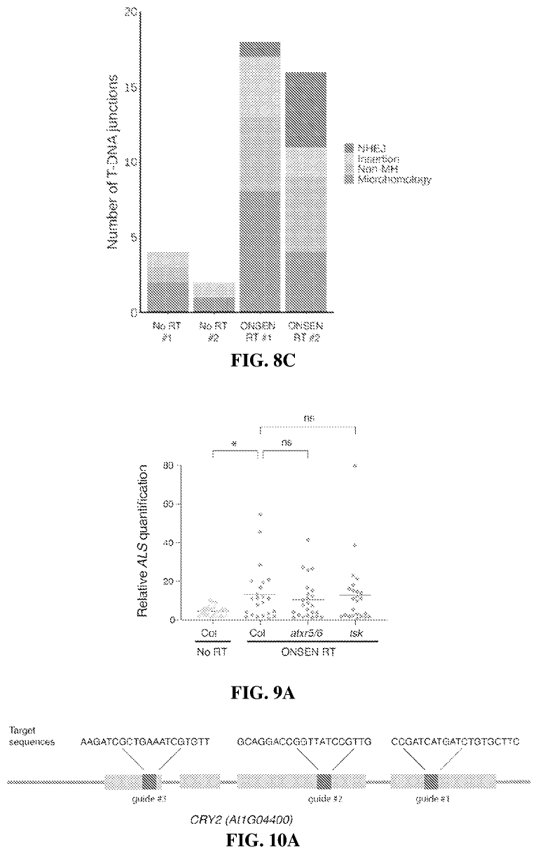

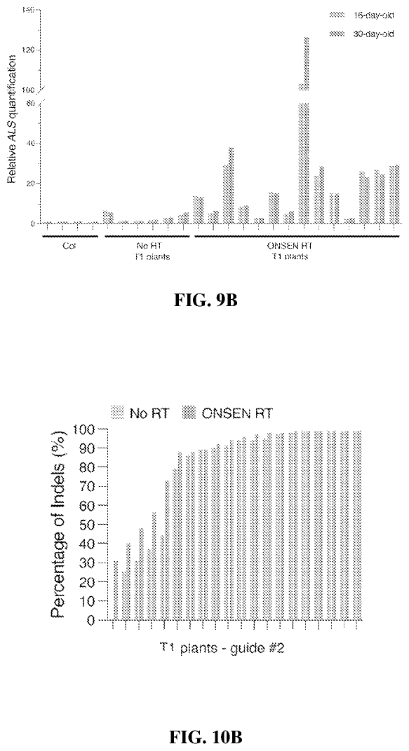

T-DNA concatenation may originate in Agrobacterium or arise before, during or after T-DNA integration in the plant genome. The results herein indicate that T-DNA levels are similar in cultured Agrobacterium strains carrying plasmids with or without RT sequences (FIG. 3A), arguing that T-DNA amplification takes place within the plant. Furthermore, whole-genome sequencing analysis did not reveal copy number variations for the genomic regions flanking the T-DNA insertion sites (FIG. 3B), suggesting that amplification occurs at the time of, or before, T-DNA integration. In Arabidopsis, two DNA repair pathways contributes to T-DNA integration: TMEJ (theta-mediated end joining) and non-homologous end joining (NHEJ)^{16,17}. TMEJ is an error-prone repair pathway responsible for inducing large (>5 kb) tandem genomic amplifications¹⁸, as observed in Arabidopsis mutants lacking the histone mark H3.1K27me1 (e.g., atxr5 atxr6 mutant^{19,20} that are characterized by amplification of heterochromatin in a manner dependent on TMEJ and the replication fork-repair factor TONSOKU (TSK)¹⁷. To test if RT-mediated T-DNA amplification is caused by specific DNA repair pathways, binary plasmids (with and without ONSEN) were transformed into different mutant backgrounds. First, NHEJ was tested by transforming ku70, ku80 and lig4 mutants. No significant effect on T-DNA amplification was detected (FIG. 3C). To assess TMEJ, studies could not directly verify the involvement of DNA polymerase theta (the main component of this repair pathway; encoded by the POLQ/TEBICHI gene), as T-DNA integration is abolished in the absence of this protein¹⁷; transformed plants were not obtained using floral dip in the absence of this protein, even when using the hypomorphic polq mutant teb-3. Therefore, the created plasmids were then transformed into three mutant backgrounds (ku70, ku80 and lig4) impaired in NHEJ, and similarly to rad51, a significant effect on T-DNA amplification was not detected (FIG. 3D). Because T-DNA integration is abolished in the absence of DNA polymerase theta protein (the main component of TMEJ)²⁰, it is challenging to directly verify the involvement of DNA polymerase theta protein. Therefore, mutant backgrounds of TMEJ-associated RAD17 and MRE11^{16,21} were assed, and a strong suppression of T-DNA amplification was observed(FIG. 3D). RAD17 and MRE11 also contribute to homologous recombination (HR)-mediated repair²²⁻²⁴, but, using a rad51 mutant background, the data showed that HR is not involved in the induction of T-DNA amplification (FIG. 3E). In support of the involvement of TMEJ in inducing T-DNA amplification, mutational signatures consistent with this repair pathway (e.g., microhomology-associated deletions and small insertions¹⁸) in sequencing reads spanning T-DNA copy junctions were also identified (FIGS. 8A-8C). Further investigations of DNA repair proteins revealed that the damage-induced kinase ATR, but not ATM, is required to increase T-DNA copy number (FIG. 3F). Finally, in contrast to heterochromatin amplification in the absence of H3.1K27me1, T-DNA amplification is not affected by mutations in ATXR5/ATXR6 or TSK, thus suggesting that the amplification mechanism is not dependent on DNA replication (FIG. 9A). In accordance, T-DNA levels are relatively constant in tissues of T1 plants separated by large number of replication cycles (FIG. 9B). In sum, the results support a key role for TMEJ in inducing T-DNA amplification before or during of T-DNA integration.

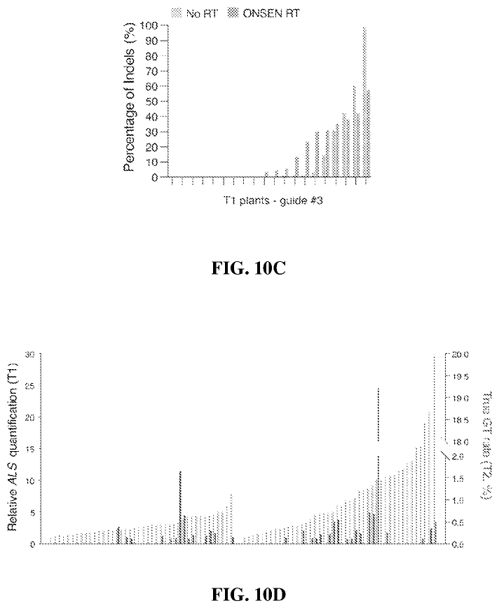

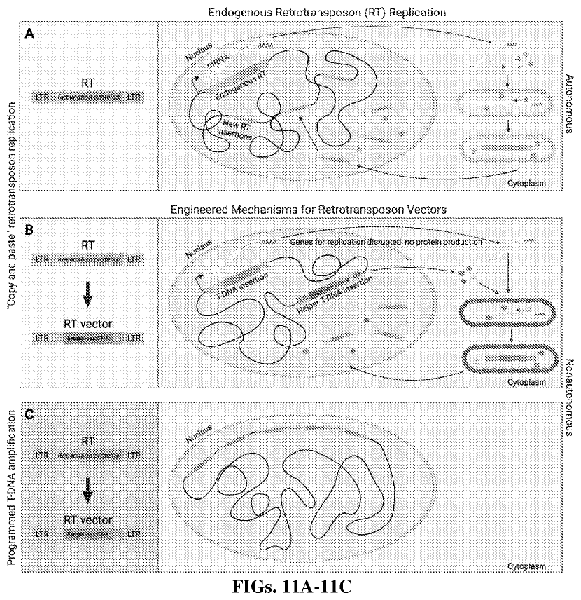

DNA repeat-mediated T-DNA concatenation allows one to increase the number of T-DNA copies in Arabidopsis transformants. Higher copy number of T-DNA has the potential to impact diverse biotechnological applications in plants. To provide a proof-of-concept of the utility of programmed T-DNA amplification, the efficiency of targeted mutagenesis by CRISPR/Cas9 using RT-derived plasmids. Three different sgRNAs that target different CRYPTOCHROME 2 (CRY2) gene of Arabidopsis were designed and tested (FIGS. 10A-10D). The data indicated that all three sgRNAs induced higher mutation rates, on average, when present on plasmids containing RT sequences (FIG. 4A and FIGS. 10A-10C). In addition, individuals with CRISPR/Cas9-mediated indels displayed higher levels of T-DNA copies than plants with no detectable indel (FIG. 10D). Taken together, these results demonstrate the benefits of inducing T-DNA amplification in Arabidopsis for increasing targeted mutagenesis rates.

Increasing T-DNA concatenation levels may also improve currently available methods used to perform gene targeting in plants. For example, in planta gene targeting (IPGT) relies on the chromosomal integration of a T-DNA containing Cas9, two sgRNAs genes, and a repair template (FIG. 4B)². One sgRNA is used to create a double-stranded DNA break to initiate DNA repair at a target locus, while the other directs Cas9 to the T-DNA to excise the repair template, which facilitates homology-directed repair (HDR). For single-copy T-DNA integrations, IPGT must rely on only one repair template copy per diploid cell in T1 plants. By inducing T-DNA amplification, more copies of the repair template can be made available for HDR in each cell, which may result in higher gene targeting rates (FIG. 4C). To test this hypothesis, a previously described system targeting the endogenous ALS locus of Arabidopsis¹¹ was modified. Mutating serine 653 to asparagine (S653N) in ALS confers resistance to the herbicide imazapyr (IM), thus providing a visual assay to detect and quantify gene targeting events¹¹. An IPGT plasmid was designed based on the ALS-IM system that contained ONSEN RT sequences. Col plants were transformed using the RT-based IPGT plasmid (ONSEN RT) or a standard IPGT plasmid (No RT) (FIG. 4B), and gene targeting rates were measured in T2 seed population (from individual T1 parents) grown on IM-containing plates. The results show that more T1 lines produced IM-resistant seedlings if transformed using the RT plasmid (37/48 or 77.1%) compared to the control plasmid (37/48 or 77.1%) compared to the control plasmid (26/44 or 59.1%) (FIGS. 4D-4E) and that, in general, higher T-DNA copy numbers in T1 plants produced more IM-resistant T2 seedlings (FIGS. 4E-4F). Comparing all T2 seedlings analyzed, a higher percentage of IM-resistant seedlings were detected if they were transformed with the RT plasmid (3.10% versus 0.68%) (FIG. 4E). In these experiments, plants can gain resistance to IM via gene targeting or through a process known as ectopic gene targeting (EGT). EGT occurs when part of the ALS genomic sequence is copied onto the T-DNA to generate a functional ALS S653N gene, which is subsequently integrated randomly into the genome¹¹. Using the ALS-IM system, EGT can easily be differentiated from true gene targeting events by PCR¹¹, and the results in the current study indicate that the frequency of true gene targeting events is approximately three times higher when the ONSEN RT plasmid is used (FIGS. 4E and 10E). Overall, these results indicate that T-DNA-dependent gene targeting systems in plants can be improved by inducing T-DNA concatenation.

### SUMMARY

In conclusion, this study revealed previously unidentified mechanisms that regulate final T-DNA structure in Arabidopsis. The ability to regulate T-DNA copy number should allow plant biologists to better control expression levels of transgenes delivered from Agrobacterium, which are subject to RNAi-mediated silencing in plants³. It is interesting to note that many binary plasmids used for Agrobacterium-mediated transformation contain DNA direct repeats. For example, the 35S promoter, commonly used in T-DNA to drive constitutive expression of a gene-of-interest or a selection marker in T-DNA, frequently includes two tandem repeated copies of an enhancer sequence²⁶. Therefore, re-designing standard binary plasmids to avoid DNA repeats may generate more phenotypic consistency when comparing individual T1 lines generated from Agrobacterium-mediated transformation, or between plant generations. Conversely, using RT or other DNA repeats on T-DNA provides an opportunity to increase the amount of exogenous DNA integrated at a single locus in plant genomes, which can contribute to useful applications in plant biology as demonstrated in this study with gene editing.

### REFERENCES

1. Chilton, et al. Cell 11, 263-271, doi: 10.1016/0092-8674(77)90043-5 (1977).
2. Galbiati, et al., Genomics 1, 25-34, doi:10.1007/s101420000007 (2000).
3. Jupe, et al. PLoS Genet 15, e1007819, doi:10.1371/journal.pgen.1007819 (2019).
4. Zambryski, et al. Science 209, 1385-1391, doi: 10.1126/science.6251546 (1980).
5. Baltes, et al. Plant Cell 26, 151-163, doi:10.1105/tpc.113.119792 (2014).
6. Jacob, et al. Nature 466, 987-991, doi:10.1038/nature09290 (2010).
7. Davarinejad, et al. Science 375, 1281-1286, doi:10.1126/science.abm5320 (2022).
8. Zaratiegui, Replication. Viruses 9, doi:10.3390/v9030057 (2017).
9 Cavrak, et al. PLoS Genet 10, e1004115, doi:10.1371/journal.pgen.1004115 (2014).
10 Ito, et al. Nature 472, 115-119, doi:10.1038/nature09861 (2011).
11 Wolter, et al. Plant J 94, 735-746, doi:10.1111/tpj.13893 (2018).
12 Bechtold, et al. Comp. Rend. L'Acad. des Sci. Serie III 316, 1194-1199 (1993).
13 Alonso, et al. Science 301, 653-657, doi:10.1126/science.1086391 (2003).
14 McElver, et al. Genetics 159, 1751-1763, doi:10.1093/genetics/159.4.1751 (2001).
15 Sessions, et al. Plant Cell 14, 2985-2994, doi:10.1105/tpc.004630 (2002).
16 Kralemann, et al. Nat Plants 8, 526-534, doi:10.1038/s41477-022-01147-5 (2022).
17 van Kregten, et al. Nat Plants 2, 16164, doi:10.1038/nplants.2016.164 (2016).
18 Kamp, Nat Commun 11, 3615, doi:10.1038/s41467-020-17455-3 (2020).
19 Jacob, et al. Science 343, 1249-1253, doi:10.1126/science.1248357 (2014).
20 Jacob, et al. Nat Struct Mol Biol 16, 763-768, doi:10.1038/nsmb.1611 (2009).
21 Hussmann, et al. Cell 184, 5653-5669 e5625, doi:10.1016/j.cell.2021.10.002 (2021).
22 Budzowska, et al. Embo J 23, 3548-3558, doi:10.1038/sj.emboj.7600353 (2004).
23 Wang, et al. Embo J 33, 862-877, doi:10.1002/embj.201386064 (2014).
24 Williams, et al. DNA Repair (Amst) 9, 1299-1306, doi:10.1016/j.dnarep.2010.10.001 (2010).
25 Fauser, et al. Proc Natl Acad Sci USA 109, 7535-7540, doi:10.1073/pnas.1202191109 (2012).
26 Kay, et al. Science 236, 1299-1302, doi:10.1126/science.236.4806.1299 (1987).
27 Brzezinka, et al Plant Cell Environ 42, 771-781, doi:10.1111/pce.13365 (2019).
28 Li, et al. Proc Natl Acad Sci USA 101, 10596-10601, doi: 10. 1073/pnas.0404110101 (2004).
29 Valuchova, et al. Plant Cell 29, 1533-1545, doi:10.1105/tpc.17.00064 (2017).
30 Heacock, et al. Nucleic Acids Res 35, 6490-6500, doi:10.1093/nar/gkm472 (2007).
31 Heitzeberg, et al. Plant J 38, 954-968, doi:10.1111/j.1365-313X.2004.02097.x (2004).
32 Samanic, et al PLoS One 8, e78760, doi:10.1371/journal.pone.0078760 (2013).
33 Feng, et al. Proc Natl Acad Sci USA 114, 406-411, doi:10.1073/pnas.1619774114 (2017).
34 Culligan, et al Plant Cell 16, 1091-1104, doi:10.1105/tpc.018903 (2004).
35 Curtis, et al Plant Physiol 133, 462-469, doi:10.1104/pp. 103.027979 (2003).
36 Clough, et al Plant J 16, 735-743, doi:10.1046/j.1365-313x.1998.00343.x (1998).
37 Livak, et al Methods 25, 402-408, doi:10.1006/meth.2001.1262 (2001).
38 Wang, et al. Science 353, 598-602, doi:10.1126/science.aaf8084 (2016).
39 Rouillard, et al. Nucleic Acids Res 31, 3057-3062, doi:10.1093/nar/gkg426 (2003).
40 Sage, et al. Methods 115, 28-41, doi:10.1016/j.ymeth.2016.12.015 (2017).
41 Schindelin, et al. Nat Methods 9, 676-682, doi:10.1038/nmeth.2019 (2012).
42 Jovtchev, et al. Cytogenet Genome Res 114, 77-82, doi:10.1159/000091932 (2006).
43 Ollion, et al. Bioinformatics 29, 1840-1841, doi:10.1093/bioinformatics/btt276 (2013).
44 Chen, et al. Bioinformatics 25, 1422-1423, doi:10.1093/bioinformatics/btpl63 (2009).
46 Altschul, et al J Mol Biol 215, 403-410, doi:10.1016/S0022-2836(05)80360-2 (1990).
47 Lamesch, et al. Nucleic Acids Res 40, D1202-1210, doi:10.1093/nar/gkr1090 (2012).
48 Li, arXiv preprint arXiv:1303.3997 (2013).
49 Katoh, et al Mol Biol Evol 30, 772-780, doi:10.1093/molbev/mst010 (2013).
50 Van Bel, et al. Nucleic Acids Res 50, D1468-D1474, doi:10.1093/nar/gkab1024 (2022).
51 Danecek, et al. Gigascience 10, doi:10.1093/gigascience/giab008 (2021).
52 Ramirez, et al. Nucleic Acids Res 44, W160-165, doi:10.1093/nar/gkw257 (2016).
53 Jasper, et al. Proc Natl Acad Sci USA 91, 694-698, doi:10.1073/pnas.91.2.694 (1994).
54 Kleinboelting, et al. Mol Plant 8, 1651-1664, doi:10.1016/j.molp.2015.08.011 (2015).
55 Feng, et al. Nucleic Acids Res 49, 5095-5105, doi:10.1093/nar/gkab299 (2021).

Those skilled in the art will recognize, or be able to ascertain using no more than routine experimentation, many equivalents to the specific embodiments of the method and compositions described herein. Such equivalents are intended to be encompassed by the following claims.

1. A transgenic plant/plant part comprising one or more exogenous nucleic acid constructs comprising DNA repeats and one or more genes.

2. The transgenic plant/plant part of claim 1 wherein the DNA repeats comprise long terminal repeats (LTR) of a retrotransposon (RT).

3. The transgenic plant of claim 1 wherein the DNA repeats comprise a nucleic acid with the same length and GC content as the LTR of a RT and/or wherein the RT is selected from the group consisting of ONSEN, Copia13, copia2l, Evade and GP3-1.

4. (canceled)

5. The transgenic plant/plant part of claim 1, (a) wherein the one or more genes are present as more than one copy as a concatemer or (b) comprising at least more than a two-fold, three-fold, four-fold, five-fold increase in copy number of one or more of the genes compared to the same plant/plant part transformed without the use of a retrotransposon-derived construct containing the one or more genes and multiple pairs of DNA repeats, e.g. a standard binary vector and/or wherein the genome plant/plant part does not comprise a helper RT.

6. (canceled)

7. (canceled)

8. The transgenic plant/plant part of claim 1, wherein: (a) the one or more genes encodes a CAS endonuclease and one or more guide polynucleotide sequences, and/or (b) the one or more genes is a disease resistant gene.

9. (canceled)

10. The transgenic plant/plant part of claim 1, wherein the plant part is selected from the group consisting of plant cuttings, cells, protoplasts, cell tissue cultures, callus (calli), cell clumps, embryos, stamens, pollen, anthers, pistils, ovules, flowers, seed, petals, leaves, stems, and roots, optionally, wherein the plant part is a seed or a seedling.

11. (canceled)

12. (canceled)

13. The transgenic plant/plant part of claim 1, wherein the plant is a monocot, a dicot or an ornamental plant.

14. (canceled)

15. (canceled)

16. The transgenic plant/plant part of claim 1, wherein the one or more genes selected from the group consisting of MRE11, ATR and RAD17, has been inactivated.

17. The transgenic plant/plant part of claim 16, comprising only one copy of the gene integrated in its genome.

18. A method for enhancing transgene copy number in a transgenic plant/plant part comprising genetically engineering a plant plant/plant part to incorporate into its genome, one or more exogenous nucleic acid constructs comprising DNA repeats and one or more genes.

19. The method of claim 18 wherein the DNA repeats comprise long terminal repeats (LTR) of a retrotransposon (RT), optionally, wherein the RT is selected from the group consisting of ONSEN, Copia13, Copia2l, Evade and GP3-1.

20. The method of claim 18 wherein the DNA repeats comprise a nucleic acid with the same length and GC content as the LTR of a RT.

21. (canceled)

22. The method of claim 20, wherein incorporation of the one of more constructions results in incorporation of the one or more genes as more than one copy as a concatemer into the plant/plant part genome.

23. The method of claim 18, wherein the plant/plant part comprise least more than a two-fold, three-fold, four-fold, five-fold increase in copy number of the one or more genes compared to the same plant/plan part transformed without the use of a construct with the one or more genes and multiple pairs of DNA repeats.

24. The method of claim 18, comprising agrobacterium mediated transformation.

25. The method of claim 18, wherein the plant is a monocot.

26. The method of claim 18, wherein the plant is a dicot.

27. The method of claim 18, wherein the plant is an ornamental plant.

28. The method of claim 18, wherein the one or more genes selected from the group consisting of MRE11, ATR and RAD17 in the plant has been inactivated.

29. The transgenic plant/plant part of claim 28, wherein the transgenic plant comprises only one copy of the gene integrated in its genome.


SEQUENCE LISTING (37 sequences)

SEQ ID NO: 1 - DNA, 440 bp, Arabidopsis thaliana
  source 1..440: mol_type = genomic DNA
agtatgtgtttttccttcaagaggtatgtgggtgcgtgaacaaatgaatagtatacgtatttactcgatgtgtttagtttctcatatttctcctggaaattaaaaaaatattttatatccaaatgaaaaaccgttttacggaatgaatctacattattacctgtataaaaaaatcaatatagctaaggacaaaagcgactttaaattctaattattgattttagcgaaaaagtttcatttagaaagcagatattgattgacacgatttaatagaacgtttgaggattagattaaattaaatagtttaatattgatatatctggctttaaaatttagtatagtatattaatcggagacgaattaaaaacacaaattttcaaaatcaaacgagctcattacatcaattaattttgatatattatgtgaacaatgttttaaag

SEQ ID NO: 2 - DNA, 1352 bp, Arabidopsis thaliana
  source 1..1352: mol_type = genomic DNA
gaataaaagcatttgcgtttggtttatcattgcgtttatacaaggacagagatccactgagctggaatagcttaaaaccattatcagaacaaaataaaccattttttgttaagaatcagagcatagtaaacaacagaaacaacctaagagaggtaacttgtccaagaagatagctaattatatctattttataaaagttatcatagtttgtaagtcacaaaagatgcaaataacagagaaactaggagacttgagaatatacattcttgtatatttgtattcgagattgtgaaaatttgaccataagtttaaattcttaaaaagatatatctgatctagatgatggttatagactgtaattttaccacatgtttaatgatggatagtgacacacatgacacatcgacaacactatagcatcttatttagattacaacatgaaatttttctgtaatacatgtctttgtacataatttaaaagtaattcctaagaaatatatttatacaaggagtttaaagaaaacatagcataaagttcaatgagtagtaaaaaccatatacagtatatagcataaagttcaatgagtttattacaaaagcattggttcactttctgtaacacgacgttaaaccttcgtctccaataggagcgctactgattcaacatgccaatatatactaaatacgtttctacagtcaaatgctttaacgtttcatgattaagtgactatttaccgtcaatcctttcccattcctcccactaatccaactttttaattactcttaaatcaccactaagctagtaacgcctatcatgaattagctctactaaatctagcaacctttcaaatttgcagtattgcaggtgtctctgtgtctttaaaatagttgccttatgatttcttcggtttcaagatgatcaaatagttatagatttcatgctcacacatgctcattagatgtgtacatactttacttacccaaatctattttctcgcaaagattttgatggtaaagctgatttggttctattgaactaaatcaaacgagtttcagactgagtgattctaatccggcccattagcccctaaacagacccactaattacgcagcttttaatagagtaattacacctagtttacccactaaaccactaagcactaattatctcacaatctaatgagcttccctcgtaattacttgggctttcactctaccatttatttgtaacagtcaagtctctactgtctctatataaactctctaaagttaacacacaattctcatcacaaacaaatcaaccaaagcaacttctactctttcttctttcgaccttatcaatctgttgagaa

SEQ ID NO: 3-4 - skipped (2 entries)

SEQ ID NO: 5 - DNA, 20 bp, synthetic construct
  source 1..20: mol_type = other DNA
aagatcgctgaaatcgtgtt

SEQ ID NO: 6 - DNA, 20 bp, synthetic construct
  source 1..20: mol_type = other DNA
gcaggaccggttatccgttg

SEQ ID NO: 7 - DNA, 20 bp, synthetic construct
  source 1..20: mol_type = other DNA
ccgatcatgatctgtgcttc

SEQ ID NO: 8 - DNA, 11 bp, synthetic construct
  source 1..11: mol_type = other DNA
caggatatatt

SEQ ID NO: 9 - DNA, 21 bp, synthetic construct
  source 1..21: mol_type = other DNA
tcgtggtggtgagtttgttac

SEQ ID NO: 10 - DNA, 20 bp, synthetic construct
  source 1..20: mol_type = other DNA
cagcatcatcacaagcatcc

SEQ ID NO: 11 - DNA, 19 bp, synthetic construct
  source 1..19: mol_type = other DNA
gctggctttgcaagggatg

SEQ ID NO: 12 - DNA, 21 bp, synthetic construct
  source 1..21: mol_type = other DNA
ccatcagtcaactcatcaagg

SEQ ID NO: 13 - DNA, 20 bp, synthetic construct
  source 1..20: mol_type = other DNA
ccggctcgtatgttgtgtgg

SEQ ID NO: 14 - DNA, 23 bp, synthetic construct
  source 1..23: mol_type = other DNA
ctggtgtgtgggcaatgaaactg

SEQ ID NO: 15 - DNA, 20 bp, synthetic construct
  source 1..20: mol_type = other DNA
ggtggagcacgacactctcg

SEQ ID NO: 16 - DNA, 22 bp, synthetic construct
  source 1..22: mol_type = other DNA
gatggcatttgtaggtgccacc

SEQ ID NO: 17 - DNA, 21 bp, synthetic construct
  source 1..21: mol_type = other DNA
gcctagcctaaagccaatctc

SEQ ID NO: 18 - DNA, 20 bp, synthetic construct
  source 1..20: mol_type = other DNA
gcgtgtccaagaattgcgcc

SEQ ID NO: 19 - DNA, 22 bp, synthetic construct
  source 1..22: mol_type = other DNA
atccgttggtggatgccggaat

SEQ ID NO: 20 - DNA, 20 bp, synthetic construct
  source 1..20: mol_type = other DNA
cacacatctacatgttaggc

SEQ ID NO: 21 - DNA, 24 bp, synthetic construct
  source 1..24: mol_type = other DNA
gttgttaactagagcttggtctcc

SEQ ID NO: 22 - DNA, 25 bp, synthetic construct
  source 1..25: mol_type = other DNA
catggatgatggatccattcagttg

SEQ ID NO: 23 - DNA, 22 bp, synthetic construct
  source 1..22: mol_type = other DNA
cacagtctttgattcgaagatc

SEQ ID NO: 24 - DNA, 22 bp, synthetic construct
  source 1..22: mol_type = other DNA
ggtcccgaactaacgaaacagg

SEQ ID NO: 25 - DNA, 19 bp, synthetic construct
  source 1..19: mol_type = other DNA
agtacgttgatggggctgg

SEQ ID NO: 26 - DNA, 24 bp, synthetic construct
  source 1..24: mol_type = other DNA
ccaatgtctgattagtgcttctgg

SEQ ID NO: 27 - DNA, 2340 bp, synthetic construct
  source 1..2340: mol_type = other DNA
gagagatgaaaccggtgattatcagaacctgatccgattggaaccgtcccaagcgttgcgtcataacggaaggagatggccggattaaatgatccgattggaaccgtcccaagcgttgcgatgtgatttgtccgcaccaagaacatgtgtgatccgattggaaccgtcccaagcgttgcgcaatgctggatacaccaggaccttacctgtgatccgattggaaccgtcccaagcgttgcgcaaagaaagcagatctccgagaagctattcgatccgattggaaccgtcccaagcgttgcgaggacgagatattcccgaacatgttgctgtgatccgattggaaccgtcccaagcgttgcggagctcacacatttctcggggatccggctcgatccgattggaaccgtcccaagcgttgcggttatgcaatgggaagatcggttctacaaagatccgattggaaccgtcccaagcgttgcggtacttttattaaacaaccagcatcttggcgatccgattggaaccgtcccaagcgttgcgctagccactattcgtgtagagaatcttccagatccgattggaaccgtcccaagcgttgcgggagatggaagctttataatgaatgtgcaagatccgattggaaccgtcccaagcgttgcggctaaccctgatgcgatagttgtggatattgatccgattggaaccgtcccaagcgttgcgtttggacttcctgctgcgattggagcgtctgatccgattggaaccgtcccaagcgttgcgtggctatcatcaggaggccttggagctatggatccgattggaaccgtcccaagcgttgcggcgcagttctacaattacaagaaaccaagggatccgattggaaccgtcccaagcgttgcgagtactggtgtcgggcaacatcaaatgtgggatccgattggaaccgtcccaagcgttgcgcttgatgagttgactgatggaaaagccatagatccgattggaaccgtcccaagcgttgcggaagctattcctccacagtatgcgattaaggatccgattggaaccgtcccaagcgttgcgcagaagtttccgttgagctttaagacgtttgatccgattggaaccgtcccaagcgttgcgggagtttggaggaatgagttgaacgtacaggatccgattggaaccgtcccaagcgttgcggagaaccgagcggaggagcttaagcttgatgatccgattggaaccgtcccaagcgttgcgaagctggctttgcaagggatgaataaggttgatccgattggaaccgtcccaagcgttgcgaagactcctcatgtgtctgtgtgtggtgatgatccgattggaaccgtcccaagcgttgcgattgatattgactcggctgagattgggaaggatccgattggaaccgtcccaagcgttgcggaggcttttgctagtagggctaagattgttgatccgattggaaccgtcccaagcgttgcggtaaggtttgatgatcgtgtcacgggtaaggatccgattggaaccgtcccaagcgttgcgtacgctgtggagcatagtgatttgttgttggatccgattggaaccgtcccaagcgttgcgcttttggttcattttaaccttctgtaaacagatccgattggaaccgtcccaagcgttgcggaaatagggtaattcaaaatctagcttgatgatccgattggaaccgtcccaagcgttgcgctgtgtccacattatcagttttgtgtatacgatccgattggaaccgtcccaagcgttgcgtaaaactacgatgtcatcgagaagtaaaatgatccgattggaaccgtcccaagcgttgcgtgtgctcatatgggccgtggtttccaaattgatccgattggaaccgtcccaagcgttgcggaaaatgctcttaccattggtttttaattggatccgattggaaccgtcccaagcgttgcggtcactgggttaatatctctcgaatcttgcgatccgattggaaccgtcccaagcgttgcgaattcgttattagggttctaagctgttttagatccgattggaaccgtcccaagcgttgcgattgcgaaatgcgaatggtaaattgagtaagatccgattggaaccgtcccaagcgttgcgcggtttactccttgtgactggctcagtttggatccgattggaaccgtcccaagcgttgcgctgctttttggtttacgtcagactactactgatccgattggaaccgtcccaagcgttgcgggtaatttgagtttcttttagttgttgatcgatccgattggaaccgtcccaagcgttgcg

SEQ ID NO: 28 - DNA, 1740 bp, synthetic construct
  source 1..1740: mol_type = other DNA
caagaccgacctgtccggtgccctgaatgacccatgatcgtccgatctggtcggatttgtatggacccccacccacgaggagcatcgtggcccatgatcgtccgatctggtcggatttgtcgatgcctgcttgccgaatatcatggtggacccatgatcgtccgatctggtcggatttgtaaaacgtccgcaatgtgttattaagttgtccccatgatcgtccgatctggtcggatttgttcggcgttaattcagtacattaaaaacgtccccatgatcgtccgatctggtcggatttgtcccgaattaattcggcgttaattcagtacacccatgatcgtccgatctggtcggatttgtcacctaaagtccctatagatcccccgaattcccatgatcgtccgatctggtcggatttgtaaaatccagatcacctaaagtccctatagacccatgatcgtccgatctggtcggatttgtcctaaaaccaaaatccagtactaaaatccacccatgatcgtccgatctggtcggatttgttcacgtgttgagcatataagaaacccttagcccatgatcgtccgatctggtcggatttgttagggttcttatagggtttcgctcacgtgtcccatgatcgtccgatctggtcggatttgtagtagttcccagataagggaattagggttccccatgatcgtccgatctggtcggatttgtaataatgtgtgagtagttcccagataagggcccatgatcgtccgatctggtcggatttgtgatcgacatcgagtttctccataataatgtcccatgatcgtccgatctggtcggatttgttagctagagtcgatcgacatcgagtttctccccatgatcgtccgatctggtcggatttgttctggggttcggatcgatcctctagctagacccatgatcgtccgatctggtcggatttgtctgagcgggactctggggttcggatcgatccccatgatcgtccgatctggtcggatttgtatcgccttcttgacgagttcttctgagcggcccatgatcgtccgatctggtcggatttgtcatcgccttctatcgccttcttgacgagttcccatgatcgtccgatctggtcggatttgttcgccgctcccgattcgcagcgcatcgcctcccatgatcgtccgatctggtcggatttgtgctttacggtatcgccgctcccgattcgcacccatgatcgtccgatctggtcggatttgtaatgggctgaccgcttcctcgtgctttacgcccatgatcgtccgatctggtcggatttgtgcttggcggcgaatgggctgaccgcttcctcccatgatcgtccgatctggtcggatttgtctacccgtgatattgctgaagagcttggcgcccatgatcgtccgatctggtcggatttgtcatagcgttggctacccgtgatattgctgacccatgatcgtccgatctggtcggatttgtgtgtggcggaccgctatcaggacatagcgtcccatgatcgtccgatctggtcggatttgttggccggctgggtgtggcggaccgctatcacccatgatcgtccgatctggtcggatttgtgcttttctggattcatcgactgtggccggccccatgatcgtccgatctggtcggatttgtaatatcatggtggaaaatggccgcttttctcccatgatcgtccgatctggtcggatttgt

SEQ ID NO: 29 - DNA, 30 bp, synthetic construct
  source 1..30: mol_type = other DNA
  misc_feature 1: note = Alex Fluor 488 conjugated to 5 prime Cytosine
cgcaacgcttgggacggttccaatcggatc

SEQ ID NO: 30 - DNA, 30 bp, synthetic construct
  source 1..30: mol_type = other DNA
  misc_feature 1: note = ATTO 590 conjugated to 5 prime Adenine
acaaatccgaccagatcggacgatcatggg

SEQ ID NO: 31 - DNA, 158 bp, Arabidopsis thaliana
  source 1..158: mol_type = genomic DNA
tattgggccatatgtatggaccttatatgttttgggctttaactgtaacttatcgatgaagtctcataaacccttgtgtaaaccctagcagctttgtgtatataagcaattgtatgaatgatcaataaagcaagtcagttcgatatctaattctacat

SEQ ID NO: 32 - DNA, 119 bp, Arabidopsis thaliana
  source 1..119: mol_type = genomic DNA
tataaggagttgtatatattaaggatataattgtcattagtagttgtgtataccctagactaagtactatatatgttgtaacacattcatcataataagacaattcactctcttatcac

SEQ ID NO: 33 - DNA, 406 bp, Arabidopsis thaliana
  source 1..406: mol_type = genomic DNA
tattgatcaagactcaaataagaaaggcctagtattggatatgtactacaagagagtgggccgaacatatgagaagtctatgaagagcttctagaagaggtgaaggcacacaaatatctcttgtagccgttgaagaggactcacgagtgtctttagcaacaaggtcaaccaatgaccagtcactctcatcaccaagaccaaggaacctcttctcttatgtctctttcagtcttacgttaattgtaaacagccatattctctttgtgtgtctttaaaagcattgtaaacacacaaaggttactatctaattctatcaatatgatttggtctcatattctctcacatacaaactctcttgttcttcataactctcttaatctttaatacaattccgcatattctttca

SEQ ID NO: 34 - DNA, 384 bp, Arabidopsis thaliana
  source 1..384: mol_type = genomic DNA
tgatacaggattaacgtgtggtgagctggtgagctgacgagctggcgagctggcaatgtgaagtcggatgttaggcaaagtacttgtgagctggcgagctggcgagctggcaatgcaatggacgttaacggcgatcgttattaacgtggttaacgtttactttctttacttttttatataagagcaacatgttatgctcatgctttcattatcgaataatctgtaatcttcagaattctctagaacctttgtaagtgctagatttctctcacatcgagagctagctgaatcatttggtgttttatgaacatcgattgattgttggtttcgaatgataattggagatttggattgttcttgtgaatcgcaggtttccctgtaaca

SEQ ID NO: 35 - protein, 236 aa, Arabidopsis sp.
MKYIFLLSAFLIFLFSAFIYFFPIFGAVVCPPCFGFVEVEGGIYIDASLNADLEKDKILSNLDAAHLLLREVYGEVEAPLPAIFLCVSKNCATYLGRRGEKASSFGHWAIVVYRDGNNSGILAHELSHIEIGFRLGFYNMSSVPIWFDEGVAVVASQDRRYLNVDPSGRLSCKEGVSGAVIADLDEWWRRASIGDVGIYSAAACEVMKWMDRRGNEGSLVRLLDLLRSGESFDVAF

SEQ ID NO: 36 - DNA, 635 bp, unidentified
  source 1..635: mol_type = other DNA
agagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaac

SEQ ID NO: 37 - DNA, 1357 bp, unidentified
  source 1..1357: mol_type = other DNA
taatctcttgttgctttaaattatttcatattgtaaattactttctgctttatcggttttaccatttcgggagtcttttttgtgtgcaatctgtttcgtttggtaagcttgtagtttcatgaaagtgaatgtaagatatgcattacgtttgttgctgaagtgaatgtaagatacgcactattatatctcatgattttctaagaaaaccctcttaaaacgaagatgtctatagcattacgtttctatttccatataatacgttaaaatttatggtttttacgtataaaatgcaaaataaagacacaagtatatctccaaagcaatgtaccgttgggaaaatttattagtacgttttcaattgtcaatgcaaataattaatggatgtgatagtcacaattaaacatacaataataaaaatgatgatgatgattcgatgatgtggtgggaaggataaattaaaccgactttggggcagtgacaggcagtgtcagtgtcaaagacaaccatttgtagtcactatttctatcgaaggttgcaaattgaatggtggaggagtatcaaaacgacacacatacttgaaaagatattttaataatataaaaaaattggtgatggcgtaataacaaacctagagctaattattatccttaatgataccaaatctatatgatacgatatttgttttaaaaagagtaaagactgacacttgagatgtgacactggcgatttcgctcacgtcaccacttttcccacctcaaataacgcttacggctttatccattaattctaagtataattttaagtgtattttttcttgccaaattcaaatatatcttactaaatggatgaacattataaaattgttatcaaaaccattaaatgttcttataatttctttcgttcctccaatgtcatcccaagactttttgacctaatatatgatatatctaacttgctttggaatcgtatgacatatatcttcaaatacatatttcgtatttttttttcacgaaaactaatttagaaagtagaaaaccagctattttaaagaaaataaagtgtgtttatatatattctaaaacaatgctataagaacataagaccaagatatatacaatgttattttatatttattattaagcattaacattgaaattaaaaatattaaacatgtataccaaagtaatcaacattgtagttattactactctctctgttcatttttgtttgattgtttagaaaaaacacacatattaagaaaacatattaaatattgattataaatgtattatttttaatgttttacagttttctataactttaaaccaatgataattaactatttttttaaaaaatt